SEQUENCE LISTING (3 sequences)

SEQ ID NO: 1 - DNA, 401 bp, Homo sapiens
tcaatgaactgtccctcagggatggccactgggttcaggatgcgatacctgaggagggaagtgtccagagtcacccatgctctgcagtgatctgctctgcccacagaattccagggtcacctgcagatcccattcccatcagggcagcctggctccccctgttctatgagctctggggcaccctcatacccacctctgccrgaagtccccgtagaggatgcggttggggaagcccttcctgcagatgcggatgccctccagcacgccattgcagcgcagctggtgcatgaccagggggttgtccatcacccctgtgtcaggagggaaggggaaagggtcagccttagggtaaagtggatgccagctgtcctccccagactaaggtcttctcctggctcacc

SEQ ID NO: 2 - DNA, 5820 bp, Homo sapiens
atgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtctgcctacctcatggggctgaactcagctgacctgctcaaggggctgtgccaccctcgggtgaaagtgggcaacgagtatgtcaccaaggggcagagcgtgcagcaggtttactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcctgaagcccacttctccctgatccactacgccggcactgtggactacaacatcctgggctggctggaaaaaaacaaggatcctctcaacgagactgtcgtgggcttgtatcagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgtatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaagggggcagagaagctgctcagctctctggacattgatcacaaccagtacaagtttggccacaccaaggtgttcttcaaggccgggctgctggggctgctggaggaaatgagggacgagaggctgagccgcatcatcacgcgtatccaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagatgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggccgaagaagacaaggtcaacaccctgtccaagtctaaggtcaagctggagcagcaggtggatgatctggagggatccctagagcaagagaagaaggtgcgcatggacctggagcgagcaaagcggaagctggagggtgacttgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggcaacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttgtcccggcagctagaggaaaaggaggcgctaatctcgcagcttacccgtgggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtgtaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggacagggagctgattgagaccagcgagcgggtgcagctgctgcattcccagaacaccagcctcatcaaccagaagaagaagatggatgctgacctgtcccagctccagtcggaagtggaggaggcagtgcaggagtgcagaaacgccgaggagaaggccaagaaggccatcacgcatgccgccatgatggcagaggagctgaagaaggagcaggacaccagcgcccacctggagcgcatgaagaagaacatggagcagaccattaaggacctgcagcaccggctggacgaggccgagcagatcgccctcaagggcggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtga

SEQ ID NO: 3 - protein, 1939 aa, Homo sapiens [sequence omitted]

(19) United States
(12) Patent Application Publication
(10) Pub. No.: US 2013/0338012 A1
(43) Pub. Date: Dec. 19, 2013

(54) GENETIC RISK FACTORS OF SICK SINUS SYNDROME

(71) Applicants: Patrick Sulem, Reykjavik (IS); Hilma Holm, Reykjavik (IS); Daniel Gudbjartsson, Reykjavik (IS)

(72) Inventors: Patrick Sulem, Reykjavik (IS); Hilma Holm, Reykjavik (IS); Daniel Gudbjartsson, Reykjavik (IS)

(73) Assignees: ILLUMINA INC., san Diego, CA (US); deCODE Genetics ehf., Reykjavik (IS)

(21) Appl. No.: 13/988,268

(22) PCT Filed: Nov. 17, 2011
(86) PCT No.: PCT/IS2011/050013

§ 371 Date: Aug. 6, 2013

(30) Foreign Application Priority Data
Nov. 18, 2010 (IS) 8937

(51) Int. Cl. C12Q 1/68

(52) U.S. Cl. CPC C12Q 1/6883; G06F 19/345
USPC 506/2; 436/501; 506/38; 435/6.11; 435/6.12; 435/7.92; 506/16; 435/287.2; 422/69; 702/19

(57) ABSTRACT

It has been found that certain alleles of the human MYH6 gene are predictive of risk of certain conditions, including Sick Sinus Syndrome, Atrial Fibrillation, Pacemaker implantation and Thoracic aortic aneurysm, in humans. The invention provides diagnostic applications using such alleles, including methods of determining a susceptibility of Sick Sinus Syndrome and related conditions.

### INTRODUCTION

Sick sinus syndrome (SSS) or sinus node dysfunction is a common clinical disorder characterized by pathological sinus bradycardia (slow heart rate), sinus arrest and/or chronotropic incompetence (attenuated heart rate response to exercise). The syndrome comprises a wide range of electrophysiological abnormalities, including failure of sinus node and atrial impulse formation or propagation as well as susceptibility to atrial tachyarrhythmias, particularly atrial fibrillation. While encountered at any age, SSS is primarily a disease of the elderly, and is commonly secondary to other cardiac disorders when diagnosed in younger patients. Symptoms are often intermittent and/or nonspecific and include dizziness, syncope and heart failure. The only effective treatment for symptomatic and irreversible sinus node dysfunction is permanent cardiac pacing and the syndrome remains the most common indication for permanent pacemaker (PM) implantation. In the year 2002, close to 268,000 pacemakers were implanted in the United States.

Several studies have identified gene mutations in sporadic cases and kindreds with familial SSS, both with and without other concomitant cardiac conditions, mainly through candidate gene approaches. Implicated genes have been ion channel or ion channel-associated genes including SCN5A, HCN4 and ANK2 (Benson, D W et al. J Clin Invest 112:1019-28 (2003); Schulze-Bahr, E et al. J Clin Invest Circ Res 111:1537-45 (2003); Milanesi, R. et al. N Engl J Med 354:151-7 (2006); Mohler, P J et al. Nature 421:634-9 (2003)). Similarly, knockout mouse models of various ion channel and gap junction subunits have been shown to result in the SSS phenotype.

The present invention describes variants in the human MYH6 gene that confer risk of Sick Sinus Syndrome, and their use in diagnostic applications.

### SUMMARY OF THE INVENTION

The present inventors have discovered that variants in the human MYH6 gene on chromosome 14q are associated with Sick Sinus Syndrome and related conditions. The present invention relates to the utilization of such variants in the risk management of these conditions.

In a first aspect, the invention provides a method of determining a susceptibility to Sick Sinus Syndrome, the method comprising analyzing nucleic acid sequence data from a human individual for at least one polymorphic marker in the human MYH6 gene, wherein different alleles of the at least one polymorphic marker are associated with different susceptibilities to Sick Sinus Syndrome in humans, and determining a susceptibility to Sick Sinus Syndrome from the nucleic acid sequence data.

In another aspect, the invention provides a method of determining a susceptibility to a condition selected from Sick Sinus Syndrome, Atrial Fibrillation, Pacemaker implantation and Thoracic aortic aneurysm, the method comprising analyzing data representative of at least one allele of the human MYH6 gene in a human individual; wherein different alleles of the human MYH6 gene are associated with different susceptibilities to the condition in humans, and determining a susceptibility to the condition for the human individual from the data.

Another aspect relates to a method of determining whether an individual is at increased risk of developing Sick Sinus Syndrome, the method comprising steps of obtaining a biological sample containing nucleic acid from the individual; determining, in the biological sample, nucleic acid sequence about the MYH6 gene; and comparing the sequence information to the wild-type sequence of MYH6 (SEQ ID NO:2); wherein an identification of a mutation in MYH6 in the individual is indicative that the individual is at increased risk of developing Sick Sinus Syndrome.

The invention also relates to amino acid sequence data. Thus, another aspect relates to a method of determining a susceptibility to Sick Sinus Syndrome, the method comprising obtaining amino acid sequence data about at least one encoded MYH6 protein in a human individual, and analyzing the amino acid sequence data to determine whether at least one amino acid substitution predictive of increased susceptibility of Sick Sinus Syndrome is present, wherein a determination of the presence of the at least one amino acid substitution is indicative of increased susceptibility of Sick Sinus Syndrome for the individual, and wherein a determination of the absence of the at least one amino acid substitution is indicative of the individual not having the increased susceptibility.

A further aspect relates to a method of determining a susceptibility to Sick Sinus Syndrome, the method comprising obtaining amino acid sequence data about at least one encoded MYH6 protein in a human individual; and analyzing the amino acid sequence data to determine whether at least one amino acid substitution predictive of increased susceptibility of Sick Sinus Syndrome is present; wherein a determination of the presence of the at least one amino acid substitution is indicative of increased susceptibility of Sick Sinus Syndrome for the individual, and wherein a determination of the absence of the at least one amino acid substitution is indicative of the individual not having the increased susceptibility.

The invention further provides a method of identification of a marker for use in assessing susceptibility to Sick Sinus Syndrome in human individuals, the method comprising (a) identifying at least one polymorphic marker in the human MYH6 gene; (b) obtaining sequence information about the at least one polymorphic marker in a group of individuals diagnosed with Sick Sinus Syndrome; and (c) obtaining sequence information about the at least one polymorphic marker in a group of control individuals; wherein determination of a significant difference in frequency of at least one allele in the at least one polymorphism in individuals diagnosed with Sick Sinus Syndrome as compared with the frequency of the at least one allele in the control group is indicative of the at least one polymorphism being useful for assessing susceptibility to Sick Sinus Syndrome.

Further provided are prognostic methods and methods of assessing probability to treatment. Thus, a further aspect of the invention relates to a method of predicting prognosis of an individual diagnosed with Sick Sinus Syndrome, the method comprising obtaining sequence data about a human individual about at least one polymorphic marker in the human MYH6 gene, wherein different alleles of the at least one polymorphic marker are associated with different susceptibilities to Sick Sinus Syndrome in humans, and predicting prognosis of Sick Sinus Syndrome from the sequence data. Also provided is a method of assessing probability of response of a human individual to a therapeutic agent for preventing, treating and/or ameliorating symptoms associated with Sick Sinus Syndrome, comprising obtaining sequence data about a human individual identifying at least one allele of at least one polymorphic marker in the human MYH6 gene, wherein different alleles of the at least one polymorphic marker are associated with different probabilities of response to the therapeutic agent in humans, and determining the probability of a positive response to the therapeutic agent from the sequence data.

Yet another aspect provides a method of treatment of a human individual, the method comprising steps of (i) determining whether the individual has at least one symptom selected from the group consisting of (a) sinus bradycardia; (b) sinus arrest; (c) chronotrophic incompetence; (d) tachycardia; and (e) atrial fibrillation; (ii) analyzing sequence information about the individual for at least one allele of the human MYH6 gene, wherein at least one allele is predictive of increased susceptibility of Sick Sinus Syndrome in humans; wherein the individual is selected for treatment for Sick Sinus Syndrome based on the presence of at least one symptom as listed in (i) and a determination of the presence of the at least one allele.

The invention also provides kits. In one such aspect, the invention relates to a kit for assessing susceptibility to Sick Sinus Syndrome in human individuals, the kit comprising reagents for selectively detecting at least one at-risk variant for Sick Sinus Syndrome in the individual, wherein the at least one at-risk variant is a marker in the human MYH6 gene or an amino acid substitution in an encoded MYH6 protein, and a collection of data comprising correlation data between the at least one at-risk variant and susceptibility to Sick Sinus Syndrome.

Further provided is the use of an oligonucleotide probe in the manufacture of a diagnostic reagent for diagnosing and/or assessing a susceptibility to Sick Sinus Syndrome, wherein the probe is capable of hybridizing to a segment of the human MYH6 gene with sequence as given by SEQ ID NO:2, and wherein the segment is 15-400 nucleotides in length.

The invention also provides computer-implemented applications. In one such application, the invention relates to an apparatus for determining a susceptibility to Sick Sinus Syndrome in a human individual, comprising a processor and a computer readable memory having computer executable instructions adapted to be executed on the processor to analyze information for at least one human individual with respect to at least one marker in the human MYH6 gene that is predictive of susceptibility to Sick Sinus Syndrome in humans, or at least one amino acid substitution in an encoded MYH6 protein, and generate an output based on the marker or amino acid information, wherein the output comprises at least one measure of susceptibility to Sick Sinus Syndrome for the human individual.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 shows an overview of the region around R721W. The circles show −log 10 P values for association with sick sinus syndrome for imputed SNPs based on genome-wide sequencing as a function of their build 36 coordinates. The crosses show results conditional on the effect of R721W. Recombination rates are shown in centi-Morgans per Megabase (cM/Mb). Genes in the region are shown by vertical bars (exons) and horizontal lines (introns).

FIG. 3 shows the penetrance of sick sinus syndrome among carriers and non-carriers of R721W. The upper line represents the fit of the logistic model to observed penetrance of sick sinus syndrome for 10 year birth cohorts among heterozygous carriers of R721W (crosses). The lower line and crosses represent the same information for non-carriers of R721W.

FIG. 4 shows the genomic structure of the MYH6 gene (exons as vertical bars on a straight horizontal line), size of exons of the MYH6 mRNA and functional domains of the MYH6 protein.

FIG. 5 provides a diagram illustrating a system comprising computer implemented methods utilizing risk variants as described herein.

FIG. 6 shows an exemplary system for determining risk of Sick Sinus Syndrome and related conditions, as described further herein.

FIG. 7 shows a system for selecting a treatment protocol for a subject diagnosed with Sick Sinus Syndrome or a related condition, as described further herein.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is according to WIPO ST.25:

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A or G or C or T, unknown or other |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles.

Allelic identities are described herein in the context of the marker name and the particular allele of the marker, e.g., “1 SG14S1131” refers to the 1 allele of marker SG14S1131, and is equivalent to “SG14S1131 allele 1”. Furthermore, allelic codes are as for individual markers, i.e. 1=A, 2=C, 3=G and 4=T.

The term “R721W”, in the present context, refers to an arginine to tryptophan substitution at position 721 in the MYH6 protein as set forth in SEQ ID NO:3 herein. This substitution is encoded by a G to A substitution in exon 18 of the MYH6 gene (SG14S1131 (SEQ ID NO:1); genomic location 22,936,019 bp on chromosome 14 in NCBI Build 36 of the human genome assembly). The substitution is further located at position 2,161 in the cDNA sequence of human MYH6 as shown in SEQ ID NO:2 herein. As described herein, “R721W” refers to the substitution determined by analyzing the protein sequence of the human MYH6 protein or by analyzing a nucleotide sequence encoding the amino acid at position 721 in the MYH6 protein (i.e., marker SG14S1131).

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers may be characteristic of increased susceptibility (i.e., increased risk) of Sick Sinus Syndrome, as characterized by a relative risk (RR) or odds ratio (OR) of greater than one for the particular allele. Alternatively, the markers and/or haplotypes of the invention are characteristic of decreased susceptibility (i.e., decreased risk) of Sick Sinus Syndrome, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “MYH6 gene”, as described herein, refers to the gene on chromosome 14q12 encoding the alpha heavy chain subunit of cardiac myosin. This gene is sometimes also referred to as MYHC or MYHCA.

### Genetic Variation in the MYH6 Gene Conferring Risk of Sick Sinus Syndrome

The present inventors have identified a novel missense variant in the MYH6 gene that confers significantly increased risk of conditions such as Sick Sinus Syndrome (SSS), Atrial Fibrillation, Pacemaker implantation and Thoracic aortic aneurysm in humans. This missense variant (R721W, encoded by a G to A substitution at marker SG14S1131 (SEQ ID NO:1) (C to T substitution on the reverse strand)) has an allelic frequency of 0.38% in Iceland, and associates with SSS with an Odds Ratio (OR) of 12.53 and a P-value of 1.5×10⁻²⁹. The lifetime risk of being diagnosed with SSS is around 6% for non-carriers of the R721W variant compared to approximately 50% for carriers of the mutation. The variant also decreases heart rate by about 5.05 beats per minute in individuals not diagnosed with SSS.

The R721W mutation is in exon 18 of MYH6 that encodes for part of the converter domain of the protein. This domain functions as a socket for the C-terminal α-helical tail of MYH6 and plays a critical role in amplifying the structural rearrangements in the motor domain and transmitting them to the α-helical tail during movements of the myosin during contraction. Based on the PolyPhen algorithm, the R721W mutation is predicted to alter the structure of the converter domain. It is therefore contemplated that other amino acid substitutions of this domain may also alter the structure of the domain, leading to an increased risk of Sick Sinus Syndrome.

Although the MYH6 protein itself has not been directly linked to cardiac conduction, a cardiac-specific highly conserved microRNA, miR-208a, encoded by intron 27 of MYH6 in both humans and mice, has recently been shown in mice to be necessary for maintenance of normal cardiac conduction. Both gain and loss of miR-208a function were associated with conduction abnormalities including prolonged PR (first-degree atrioventricular block), second-degree atrioventricular block and atrial fibrillation. The miR-208a is also an important regulator of cardiac gene expression in response to stress^{28,29}. Evidence suggests that miR-208a is required for expression of Cx40, which is of particular interest as mice lacking Cx40 exhibit cardiac conduction abnormalities, including of both sinus node impulse formation and atrial propagation. A potential functional consequence of the R721W mutation could be an effect on mRNA stability or processing, as previously documented for both synonymous and nonsynonymous coding mutations and thus directly affecting the amount of miR-208a present in the heart. It is therefore contemplated that R721W or other variants in the MYH6 gene may also affect mRNA stability or processing, leading to increased susceptibility of Sick Sinus Syndrome.

The inventors have surprisingly also found that after excluding the effect of known cases of Sick Sinus Syndrome, association of the R721W variant with related conditions was detected, including Pacemaker implantation, Atrial Fibrillation and Thoracic aortic aneurysm (Table 4).

The identification of the R721W variant in MYH6 provides evidence for the role of rare variants in the development of a common complex disease. This finding has a number of important clinical and diagnostic applications, as described in more detail in the following.

### Methods of Determining Susceptibility to Sick Sinus Syndrome and Related Conditions

Accordingly, the present invention provides a method of determining a susceptibility to a condition selected from the group consisting of Sick Sinus Syndrome, Pacemaker implantation, Atrial Fibrillation and Thoracic aortic aneurysm, the method comprising analyzing data from a human individual for at least one allele of the human MYH6 gene; wherein different alleles of the human MYH gene are associated with different susceptibilities to the condition in humans, and determining a susceptibility to the condition from the data.

In certain embodiments, the condition is Sick Sinus Syndrome. In certain embodiments, the condition is selected from the group consisting of Pacemaker implantation, Atrial Fibrillationa and Thoracic aortic aneurysm. In another preferred embodiment, the condition is selected from the group consisting of Atrial Fibrillation and Thoracic aortic aneurysm.

In certain embodiments, the data is nucleic acid sequence data. The nucleic acid sequence data is in certain embodiments, obtained from a biological sample containing nucleic acid from the human individual.

In certain embodiments, the at least one polymorphic marker encodes a defective MYH6 protein. The defective MYH6 protein may for example contain one or more missense substitutions, nonsense substitutions or a truncations in a MYH6 protein with sequence as set forth in SEQ ID NO:3. In one embodiment, the at least one polymorphic marker encodes a missense substitution, a nonsense substitution or a truncation in a MYH6 protein with sequence as set forth in SEQ ID NO:3.

The defective MYH6 protein may in certain embodiments have an impaired function selected from the group consisting of: an impaired converter domain, an impaired motor domain, an impaired lever arm domain, an impaired dimerization domain, an impaired alpha helix domain or an impaired myosin_N domain.

In one embodiment, the at least one allele is an allele of the polymorphic marker SG14S1131 (SEQ ID NO:1). In another embodiment, the allele is an allele one of a polymorphic marker in linkage disequilibrium with SG14S1131. In a preferred embodiment, the polymorphic marker is a marker in linkage disequilibrium with SG14S1131 characterized by values of the linkage disequilibrium measure r² of greater than 0.5. In another preferred embodiment, the at least one polymorphic marker is a marker in linkage disequilibrium with SG14S1131 characterized by values of the linkage disequilibrium measure r² of greater than 0.8.

In certain embodiment, determination of the presence of the A allele of SG14S1131 is indicative of increased risk of the condition for the individual. Determination of the absence of the A allele of SG14S1131 is indicative that the individual does not have the increased risk conferred by the allele.

Alternatively, the allele that is detected can be the allele of the complementary strand of DNA, such that the nucleic acid sequence data includes the identification of at least one allele which is complementary to any of the alleles of the polymorphic markers referenced above. For example, the allele that is detected may be the complementary T allele of the at-risk A allele of SG14S1131.

The data can be any type of data that is representative of polymorphic alleles in the MYH6 gene. In certain embodiments, the data is nucleic acid sequence data. The sequence data is data that is sufficient to provide information about particular alleles. In certain embodiments, the nucleic acid sequence data is obtained from a biological sample comprising or containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to the condition (e.g., Sick Sinus Syndrome). In certain embodiments, the sequence data is provided as genotype data, identifying the presence or absence of particular alleles at polymorphic locations.

In some embodiments, the analyzing comprises analyzing the data for the presence or absence of at least one mutant allele indicative of a MYH6 defect. The MYH6 defect may for example be a missense mutation or a nonsense mutation in MYH6, or the defect may be a premature truncation or frameshift of an encoded MYH6 protein, relative to a wild-type amino acid sequence, such as the wild-type amino acid sequence presented in SEQ ID NO:3 herein. The MYH6 defect may also be expression of a MYH6 protein with reduced or impaired activity compared to a wild-type MYH6 protein. The activity can for example be motor domain activity or dimerization activity. In one embodiment, the MYH6 defect is selected from defects that impair any of these activities.

Determination of MYH6 dimerization or MYH6 motor domain activity or other activities can be performed using standard assays well known to the skilled person. As noted above, such assays can be used to confirm that a particular MYH6 mutation impairs or eliminates a MYH6 activity and therefor would be expected to carry an increased susceptibility for a condition such as Sick Sinus Syndrome as described herein.

The data to be analyzed by the method of the invention is suitably obtained by analysis of a biological sample from a human subject to obtain information about particular alleles in the genome of the individual. In certain embodiments, the information is nucleic acid information which comprises sufficient sequence to identify the presence or absence of at least one allele in the subject (e.g. a mutant allele). The information can also be nucleic acid information that identifies at least one allele of a polymorphic marker that is in linkage disequilibrium with a mutant allele. Linkage disequilibrium may suitably be determined by the correlation coefficient between polymorphic sites. In one embodiment, the sites are correlated by values of the correlation coefficient r² of greater than 0.5. Other suitable values of r² that are also appropriate to characterize polymorphic sites in LD are however also contemplated, as discussed further herein. The information may also be information about measurement of quantity of length of MYH6 mRNA, wherein the measurement is indicative of the presence or absence of the mutant allele. For example, mutant alleles may result in premature truncation of transcribed MYH6 mRNA which can be detected by measuring the length of the MYH6 mRNA. The information may further be measurement of quantity of MYH6 protein, wherein the measurement of protein is indicative of the presence or absence of a mutant allele. Truncated transcripts will result in truncated forms of translated polypeptides, which can be measured using standard methods known in the art. For example, truncated proteins or proteins arising from a frameshift may have fewer or different epitopes from wildtype protein and can be distinguished with immunoassays. Truncated proteins or proteins altered in other ways may migrate differently and be distinguished with electrophoresis. The information obtained may also be measurement of MYH6 activity, wherein the measurement is indicative of the mutant allele. In one embodiment, the information is selected from any one of the above mentioned types of information.

In a further embodiment of the invention, a biological sample is obtained from the human subject prior to the analyzing steps. The analyzing may also suitably be performed by analyzing data from a preexisting record about the human subject. The preexisting record may for example include sequence information or genotype information about the individual, which can identify the presence or absence of mutant alleles.

In certain embodiments, information about risk for the human subject can be determined using methods known in the art. Some of these methods are described herein. For example, information about odds ratio (OR), relative risk (RR) or lifetime risk (LR) can be determined from information about the presence or absence of particular mutant alleles of MYH6.

It should be apparent from the foregoing that another aspect of the invention may relate to a method of determining whether an individual is at increased risk of developing a condition selected from Sick Sinus Syndrome, Atrial Fibrillation, Pacemaker implantation and Thoracic aortic aneurysm, the method comprising steps of (a) obtaining a biological sample containing nucleic acid from the individual; (b) determining, in the biological sample, nucleic acid sequence about the MYH6 gene, and (c) comparing the sequence information to the wild-type sequence of MYH6, as set forth in SEQ ID NO:2 herein, wherein the identification of a mutation in MYH6 in the individual is indicative that the individual is at increased risk of developing the condition.

Alternatively, the invention provides a method of determining whether an individual is at increased risk of developing a condition selected from Sick Sinus Syndrome, Atrial Fibrillation, Pacemaker implantation and Thoracic aortic aneurysm, the method comprising steps of determining, in a biological sample from the individual, nucleic acid sequence about the MYH6 gene, and comparing the sequence information to the wild-type sequence of MYH6, as set forth in SEQ ID NO:2 herein, wherein the identification of a mutation in MYH6 in the individual is indicative that the individual is at increased risk of developing the condition.

The mutation may for example be a missense mutation, a promoter mutation, a nonsense mutation or a frameshift mutation in MYH6. The mutation may further result in a MYH6 defect as described in the above.

In another aspect, the invention provides a method of determining a susceptibility to a condition selected from Sick Sinus Syndrome, Atrial Fibrillation, Pacemaker implantation and Thoracic aortic aneurysm, the method comprising analyzing sequence data from a human subject for at least one variant in the human MYH6 gene, or in an encoded human MYH6 protein, wherein different alleles of the at least one variant are associated with different susceptibilities to the condition in humans, and determining a susceptibility to the condition for the human subject from the sequence data. In a preferred embodiment, the variant is SG14S1131. In another embodiment, the variant is a variant in linkage disequilibrium with SG14S1131.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

### Methods of Determining Susceptibility to Conditions Indicative of Sick Sinus Syndrome

Sick Sinus Syndrome is characterized by certain clinical conditions, which are a result of an underlying pathological or abnormal state. The association of the R721W variant with SSS is therefore also predictive of these conditions.

As a consequence, the present invention provides a method of determining a susceptibility to a condition selected from the group consisting of sinus bradycardia, sinus arrest, chronotrophic incompetence, sinus node exit block and arrhythmia, the method comprising analyzing nucleic acid sequence data from a human individual for at least one polymorphic marker in the human MYH6 gene; wherein different alleles of the at least one polymorphic marker are associated with different susceptibilities to the condition in humans, and determining a susceptibility to the condition from the nucleic acid sequence data.

In one embodiment, the at least one polymorphic marker is SG14S1131 (SEQ ID NO:1). In another embodiment, the at least one polymorphic marker is a marker in linkage disequilibrium with SG14S1131. In a preferred embodiment, the at least one polymorphic marker is a marker in linkage disequilibrium with SG14S1131 characterized by values of the linkage disequilibrium measure r² of greater than 0.5. In another preferred embodiment, the at least one polymorphic marker is a marker in linkage disequilibrium with SG14S1131 characterized by values of the linkage disequilibrium measure r² of greater than 0.8.

In certain embodiment, determination of the presence of the A allele of SG14S1131 is indicative of increased risk of the condition for the individual. Determination of the absence of the A allele of SG14S1131 is indicative that the individual does not have the increased risk conferred by the allele.

### Obtaining Nucleic Acid Sequence Data

Sequence data can be nucleic acid sequence data, which may be obtained by means known in the art. Sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject. The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to Sick Sinus Syndrome, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the MYH6 gene, or the probe can be the complementary sequence of such a sequence. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles or haplotypes that are associated with eosinophilia, asthma, myocardial infarction, and/or hypertension.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different. Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with particular vascular conditions, as described herein.

In certain embodiments, arrays of oligonucleotide probes that are complementary to target nucleic acid sequence segments from a subject, can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a salive sample or a buccal swab.

### Protein Analysis

Missense, nonsense and frameshift nucleic acid variations may lead to an altered amino acid sequence, as compared to the non-variant (e.g., wild-type) protein, due to one or more amino acid substitutions, deletions, or insertions, or truncation (due to, e.g., splice variation). In such instances, detection of the amino acid substitution of the variant protein may be useful. This way, nucleic acid sequence data may be obtained through indirect analysis of the nucleic acid sequence of the allele of the polymorphic marker, i.e. by detecting a protein variation.

The variants described herein result in altered MYH6 protein. Accordingly, one aspect of the invention relates to a method of determining a susceptibility to a condition selected from Sick Sinus Syndrome, Atrial Fibrillation, Pacemaker implantation and Thoracic aortic aneurysm, the method comprising obtaining amino acid sequence data about at least one encoded MYH6 protein in a human individual; and analyzing the amino acid sequence data to determine whether at least one amino acid substitution predictive of increased susceptibility of Sick Sinus Syndrome is present; wherein a determination of the presence of the at least one amino acid substitution is indicative of increased susceptibility of Sick Sinus Syndrome for the individual, and wherein a determination of the absence of the at least one amino acid substitution is indicative of the individual not having the increased susceptibility. In certain embodiments, the condition is Sick Sinus Syndrome. In certain embodiments, the amino acid substitution is a nonsense mutation, a frameshift mutation or a missense mutation.

Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used. In certain embodiments of the present invention, the R721W substitution is detected in a protein sample. The detection may be suitably performed using any of the methods described in the above.

In some cases, a variant protein has altered (e.g., upregulated or downregulated) biological activity, in comparison to the non-variant or wild-type protein. The biological activity can be, for example, a binding activity or enzymatic activity. In this instance, altered biological activity may be used to detect a variation in protein encoded by a nucleic acid sequence variation. In certain embodiments, the MYH6 protein has an impaired or defective function selected from the group consisting of: impaired converter domain, an impaired motor domain, an impaired lever arm domain, an impaired dimerization domain, an impaired alpha helix domain or an impaired myosin_N domain. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, a protein variation encoded by a genetic variation could lead to an altered expression level, e.g., an increased expression level of an mRNA or protein, a decreased expression level of an mRNA or protein. In such instances, nucleic acid sequence data about the allele of the polymorphic marker, or protein sequence data about the protein variation, can be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for which a susceptibility is being determined. The biological sample can be any nucleic acid or protein containing sample obtained from the human individual. For example, the biological sample can be any of the biological samples described herein.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The markers can be independent of SG14S1131 and/or the markers may be in linkage disequilibrium SG14S1131. The polymorphic markers can be the ones of the group specified herein or they can be different polymorphic markers that are not listed herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the marker SG14S1131, encoding the R721W substitution in MYH6 may be detected directly to determine risk of Sick Sinus Syndrome. Alternatively, any marker in linkage disequilibrium with SG14S1131 may be detected to determine risk.

The present invention thus refers to the SG14S1131 and R721W markers used for detecting association to Sick Sinus Syndrome, as well as markers in linkage disequilibrium with these markers. Thus, in certain embodiments of the invention, markers that are in LD with these markers and/or haplotypes of the invention, as described herein, may be used as surrogate markers.

Suitable surrogate markers may be selected using public information, such as from the International HapMap Consortium (http://www.hapmap.org) and the International 1000genomes Consortium (http://www.1000genomes.org). The stronger the linkage disequilibrium to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fᵢ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, for example a cohort from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessary from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### Determining Risk

In the present context, an individual who is at an increased susceptibility (i.e., increased risk) for Sick Sinus Syndrome is an individual who is carrying at least one at-risk variant in the human MYH6 gene, or a variant encoded by a variation in the human MYH6 gene. In one embodiment, significance associated with a marker is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 2.0, including but not limited to: at least 3.0, at least 4.0, at least 5.0, at least 6.0, at least 7.0, at least 8.0, at least 9.0, at least 10.0, at least 11.0, at least 12.0, and at least 13.0. In a particular embodiment, a risk (relative risk and/or odds ratio) of at least 12.0 is significant. In another particular embodiment, a risk of at least 12.5 is significant.

An at-risk polymorphic marker or haplotype as described herein is one where at least one allele of at least one marker or haplotype is more frequently present in an individual at risk for the disease (SSS) (affected), or diagnosed with the disease, compared to the frequency of its presence in a comparison group (control), such that the presence of the marker is indicative of susceptibility to the disease. The control group may in one embodiment be a population sample, i.e. a random sample from the general population. In another embodiment, the control group is represented by a group of individuals who are disease-free, i.e. individuals who have not been diagnosed with the disease.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or genotype data to a database containing correlation data between polymorphic markers and susceptibility to Sick Sinus Syndrome. The database can be part of a computer-readable medium described herein.

In a specific aspect of the invention, the database comprises at least one measure of susceptibility to the condition for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to the condition for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to Sick Sinus Syndrome further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers in individuals who are members of a target population. Such a target population is in one embodiment a population or group of individuals at risk of developing Sick Sinus Syndrome, based on other genetic factors, biomarkers, biophysical parameters (e.g., ECG measures), history of SSS or related diseases, family history of SSS or a related disease).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of SSS in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (See, for example: Sulem, P., et al. Nat Genet May 17, 2009 (Epub ahead of print); Rafnar, T., et al. Nat Genet 41:221-7 (2009); Gretarsdottir, S., et al. Ann Neurol 64:402-9 (2008); Stacey, S. N., et al. Nat Genet 40:1313-18 (2008); Gudbjartsson, D. F., et al. Nat Genet 40:886-91 (2008); Styrkarsdottir, U., et al. N Engl J Med 358:2355-65 (2008); Thorgeirsson, T., et al. Nature 452:638-42 (2008); Gudmundsson, 3., et al. Nat Genet. 40:281-3 (2008); Stacey, S. N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Frayling, T M, Nature Reviews Genet. 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers described herein to be associated with risk of SSS will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as tough herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to a Sick Sinus Syndrome. The invention also provides a method of identification of a marker for use in assessing susceptibility to Sick Sinus Syndrome. The method may comprise analyzing the frequency of at least one allele of a polymorphic marker in a population of human individuals diagnosed with SSS, wherein a significant difference in frequency of the at least one allele in the population of human individuals diagnosed with SSS as compared to the frequency of the at least one allele in a control population of human individuals is indicative of the allele as a marker of the SSS. In certain embodiments, the candidate marker is a marker in linkage disequilibrium with SG14S1131.

In one embodiment, the method comprises (i) identifying at least one polymorphic marker in linkage disequilibrium, as determined by values of r² of greater than 0.5, with SG14S1131; (ii) obtaining sequence information about the at least one polymorphic marker in a group of individuals diagnosed with SSS; and (iii) obtaining sequence information about the at least one polymorphic marker in a group of control individuals; wherein determination of a significant difference in frequency of at least one allele in the at least one polymorphism in individuals diagnosed with SSS as compared with the frequency of the at least one allele in the control group is indicative of the at least one polymorphism being useful for assessing susceptibility to SSS.

In one embodiment, an increase in frequency of the at least one allele in the at least one polymorphism in individuals diagnosed with SSS, as compared with the frequency of the at least one allele in the control group, is indicative of the at least one polymorphism being useful for assessing increased susceptibility to SSS. In another embodiment, a decrease in frequency of the at least one allele in the at least one polymorphism in individuals diagnosed with SSS, as compared with the frequency of the at least one allele in the control group, is indicative of the at least one polymorphism being useful for assessing decreased susceptibility to, or protection against, SSS.

### Utility of Genetic Testing

The R721W mutation confers a very high risk of Sick Sinus Syndrome, increasing the lifetime risk of SSS from about 6% in non-carriers to about 50% for carriers. While identification of the mutation does not establish a diagnosis in itself, given the high penetrance of the mutation, its identification has significant diagnostic implications.

Particularly based on the high risk associated with this variant, the variant may be clinically useful in identifying those individuals who have intermittent or vague symptoms who also have high risk of SSS and may thus benefit from a more thorough evaluation and search for SSS, such as with long term cardiac rhythm monitoring.

Individual identified thus identified as having SSS, or symptoms consistent with an onset of SSS, could then be closely monitored or selected for immediate therapy. Common therapy for SSS is Pacemaker placement, but in some cases medical therapy is sufficient.

### Diagnostic Methods

Polymorphic markers associated with increased susceptibility of Sick Sinus Syndrome and related conditions are useful in diagnostic methods. While methods of diagnosing such conditions are known in the art, the detection of one or more alleles of the specific polymorphic markers advantageously may be useful for detection of these conditions at their early stages and may also reduce the occurrence of mis-diagnosis. In this regard, the invention further provides methods of diagnosing these conditions comprising obtaining sequence data identifying at least one allele of at least one polymorphic marker of a specified group, in conjunction with carrying out one or more steps, e.g., clinical diagnostic steps, such as any of those described herein.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a sequencing or genotyping service. The layman may also be a genotype or sequencing service provider, who performs analysis on a DNA sample from an individual, in order to provide service related to genetic risk factors for particular traits or diseases, based on the genotype status of the individual (i.e., the customer). Sequencing methods include for example those discussed in the above, but in general any suitable sequencing method may be used in the methods described and claimed herein. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications.

The application of disease-associated alleles as described herein, can thus for example be performed by the individual, through analysis of his/her genotype data, by a health professional based on results of a clinical test, or by a third party, including the genotype or sequencing service provider. The third party may also be service provider who interprets genotype or sequence information from the customer to provide service related to specific genetic risk factors, including the genetic markers described herein. In other words, the diagnosis or determination of a susceptibility of genetic risk can be made by health professionals, genetic counselors, third parties providing genotyping and/or sequencing service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available method for determining a susceptibility or risk of disease, including those mentioned above.

In certain embodiments, a sample containing genomic DNA from an individual is collected. Such sample can for example be a buccal swab, a saliva sample, a blood sample, or other suitable samples containing genomic DNA, as described further herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample). The genomic DNA obtained from the individual is then analyzed using any common technique available to the skilled person, such as high-throughput technologies for genotyping and/or sequencing. Results from such methods are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein associated with risk of Sick Sinus Syndrome. Genotype and/or sequencing data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. The calculated risk estimated can be made available to the customer via a website, preferably a secure website.

In certain embodiments, a service provider will include in the provided service all of the steps of isolating genomic DNA from a sample provided by the customer, performing genotyping or sequencing of the isolated DNA, calculating genetic risk based on the genotype or sequence data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping and/or sequencing service and interpretation of the resulting sequence data, starting from a sample of isolated DNA from the individual.

Decreased susceptibility is in general determined based on the absence of particular at-risk alleles and/or the presence of protective alleles. As discussed in more detail herein, for biallelic markers such as SNPs, the alternate allele of an at-risk allele is, by definition, a protective allele. Determinations of its presence, in particular for homozygous individuals, is thus indicative of a decreased susceptibility.

### Prognostic Methods

In addition to the utilities described above, the polymorphic markers of the invention are useful in determining a prognosis of a human individual experiencing symptoms associated with, or an individual diagnosed with a condition selected from Sick Sinus Syndrome, Atrial Fibrillation, Pacemaker implantation and Thoracic aortic aneurysm. Accordingly, the invention provides a method of predicting prognosis of an individual experiencing symptoms associated with, or an individual diagnosed with, a condition selected from Sick Sinus Syndrome, Atrial Fibrillation, Pacemaker implantation and Thoracic aortic aneurysm. The method comprises analyzing sequence data about a human individual for at least one allele of the human MYH6 gene, wherein different alleles of the MYH6 gene are associated with different susceptibilities to the condition in humans, and predicting prognosis of the individual from the sequence data. In one embodiment, the at least one polymorphic marker selected from the group consisting of SG14S1131, and markers in linkage disequilibrium therewith.

The prognosis predicted by the methods of the invention can be any type of prognosis relating to the progression of the condition, and/or relating to the chance of recovering from the condition. The prognosis can, for instance, relate to the severity of the condition, when the condition may take place (e.g., the likelihood of Pacemaker Placement being necessary), or how the condition will respond to therapeutic treatment.

With regard to the prognostic methods described herein, the sequence data can be nucleic acid sequence data or amino acid sequence data. For example, in one embodiment, determination of the presence of a frameshift mutation or a nonsense mutation in MYH6 is indicative of prognosis of ovarian cancer. The determination of the presence of a mutation in MYH6 that leads to loss of function or loss of expression of MYH6 is in certain embodiments indicative of a worsened prognosis of the condition. In other words, the presence of such mutations is in certain embodiments indicative that the individual has a worse prognosis of the condition than do individuals with the condition who do not carry such mutations. In one embodiment, determination of the presence of the R721W substitution in a protein with sequence as set forth in SEQ ID NO:3 herein is indicative of risk of the condition, and may thus also be useful in prognostic applications. Suitable methods of obtaining each are known in the art, some of which are described herein.

### Methods for Predicting Response to Therapeutic Agents

As is known in the art, individuals can have differential responses to a particular therapy (e.g., a therapeutic agent or therapeutic method). Pharmacogenomics addresses the issue of how genetic variations (e.g., the variants (markers and/or haplotypes) of the invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the invention), or therapeutic failure of the drug. Therefore, the variants of the invention may determine the manner in which a therapeutic agent and/or method acts on the body, or the way in which the body metabolizes the therapeutic agent.

Accordingly, in one embodiment, the presence of a particular allele at a polymorphic site (e.g., the SG14S1131 G->A substitution and/or the R721W substitution) is indicative of a different response, e.g. a different response rate, to a particular treatment modality. This means that a patient diagnosed with a condition such as Sick Sinus Syndrome, Atrial Fibrillation, Pacemaker implantation and Thoracic aortic aneurysm, and carrying such risk alleles would respond better to, or worse to, a specific therapeutic, drug and/or other therapy used to treat the condition. Therefore, the presence or absence of the marker allele could aid in deciding what treatment should be used for the patient. If the patient is positive for the marker allele, then the physician recommends one particular therapy, while if the patient is negative for the at least one allele of a marker, then a different course of therapy may be recommended (which may include recommending that no immediate therapy, other than serial monitoring for progression of symptoms, be performed). Thus, the patient's carrier status could be used to help determine whether a particular treatment modality should be administered.

Another aspect of the invention relates to methods of selecting individuals suitable for a particular treatment modality, based on the their likelihood of developing particular complications or side effects of the particular treatment. It is well known that many therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status (e.g., the presence or absence of the SG14S1131 G->A or R721W substitution) of the individual to decide on a suitable therapeutic procedure or on a suitable therapeutic agent to treat Sick Sinus Syndrome or a related condition. It is therefore contemplated that the polymorphic markers of the invention can be used in this manner. Indiscriminate use of a such therapeutic agents or treatment modalities may lead to unnecessary and needless adverse complications.

In view of the foregoing, the invention provides a method of assessing an individual for probability of response to a therapeutic agent for preventing, treating, and/or ameliorating symptoms associated Sick Sinus Syndrome. In one embodiment, the method comprises: analyzing nucleic acid sequence data from a human individual for at least one polymorphic marker selected from the group consisting of SG14S1131, and markers in linkage disequilibrium therewith, wherein determination of the presence of the A allele of SG14S1131, or a marker allele in linkage disequilibrium therewith, indicative of a probability of a positive response to the therapeutic agent.

In a further aspect, the markers of the invention can be used to increase power and effectiveness of clinical trials. Thus, individuals who are carriers of the R721W or SG14S1131 G->A variant, or other variants in the MYH6 gene that confer risk of Sick Sinus Syndrome, may be more likely to respond to a particular treatment modality. For some treatments, the genetic risk may correlate with less responsiveness to therapy. This application can improve the safety of clinical trials, but can also enhance the chance that a clinical trial will demonstrate statistically significant efficacy, which may be limited to a certain sub-group of the population. Thus, one possible outcome of such a trial is that carriers of the at-risk markers of the invention are statistically significantly likely to show positive response to the therapeutic agent, i.e. experience alleviation of symptoms associated with Sick Sinus Syndrome, or a related condition, when taking the therapeutic agent or drug as prescribed. Another possible outcome is that genetic carriers show less favorable response to the therapeutic agent, or show differential side-effects to the therapeutic agent compared to the non-carrier. An aspect of the invention is directed to screening for such pharmacogenetic correlations.

### Methods of Treatment

The markers found by the present inventors to confer increased risk of Sick Sinus Syndrome may be useful for establishing a diagnosis and applying an appropriate treatment. In particular, the at-risk markers for SSS(R721W; SG14S1131) may be useful for selecting individuals for suitable therapy of Sick Sinus syndrome, based on symptoms of SSS in an individual and a positive determination of the presence of an at-risk variant for SSS in the genome of the individual. In one embodiment, the therapy is by Pacemaker Placement.

As a consequence, the invention provides a method of treatment of a human individual, the method comprising (i) determining whether the individual has at least one symptom selected from the group consisting of sinus bradycardia, sinus arrest, chronotrophic incompetence, tachycardia and atrial fibrillation; (ii) analyzing sequence information about the individual for at least one polymorphic marker in the MYH6 gene, wherein at least one at-risk allele of the at least one polymorphic marker is predictive of increased susceptibility of Sick Sinus Syndrome in humans; wherein the individual is selected for treatment for Sick Sinus treatment based on the presence of at least one symptom as listed in (i) and a determination of the presence of the at least one at-risk allele. In one embodiment, the treatment is Pacemaker Placement therapy.

In one embodiment, the at least one at-risk allele of is the A allele of marker SG14S1131.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered MYH6 polypeptide encoded by a nucleic acid of the invention as described herein (e.g., the R721W variant) or to a non-altered (native) MYH6 polypeptide, means for amplification of nucleic acids, means for analyzing the nucleic acid sequence of nucleic acids, means for analyzing the amino acid sequence of a polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., dna polymerase). Additionally, kits can provide reagents for assays to be used in combination with the methods of the present invention, e.g., reagents for use with other diagnostic assays for Sick Sinus Syndrome or related conditions.

In one embodiment, the invention pertains to a kit for assaying a sample from a subject to detect a susceptibility to Sick Sinus Syndrome in the subject, wherein the kit comprises reagents necessary for selectively detecting at least one at-risk variant for Sick Sinus Syndrome in the individual, wherein the at least one at-risk variant is a marker in the human MYH6 gene or an amino acid substitution in an encoded MYH6 protein. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with the condition risk. In one such embodiment, the polymorphism is selected from the group consisting of SG14S1131, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking the polymorphism. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In one embodiment, the DNA template is amplified before detection by PCR. The DNA template may also be amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele (e.g. allele A of SG14S1131) is indicative of a increased susceptibility of Sick Sinus Syndrome. In another embodiment, determination of the presence of a marker allele is indicative of prognosis of Sick Sinus Syndrome. In another embodiment, the presence of the marker allele or haplotype is indicative of response to a therapeutic agent for Sick Sinus Syndrome. In yet another embodiment, the presence of the marker allele or haplotype is indicative of progress of treatment of Sick Sinus Syndrome.

In certain embodiments, the kit comprises reagents for detecting no more than 100 alleles in the genome of the individual. In certain other embodiments, the kit comprises reagents for detecting nor more than 20 alleles in the genome of the individual.

In a further aspect of the present invention, a pharmaceutical pack (kit) is provided, the pack comprising a therapeutic agent and a set of instructions for administration of the therapeutic agent to humans diagnostically tested for an at-risk variant for Sick Sinus Syndrome. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

The kit may additionally or alternatively comprise reagents for detecting an amino acid substitution in a human MYH6 protein, e.g., the R721W substitution. In one embodiment, the kit comprises at least one antibody for selectively detecting the R721W substitution. Other reagents useful for detecting amino acid substitutions are known to the skilled person and are also contemplated.

In certain embodiments, the kit further comprises a set of instructions for using the reagents comprising the kit. In certain embodiments, the kit further comprises a collection of data comprising correlation data between the at least one at-risk variant and susceptibility to Sick Sinus Syndrome.

### Antisense Agents

The nucleic acids and/or variants described herein, e.g. the SG14S1131 and R721W variants, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Lavery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises the human MYH6 gene. In certain other embodiments, the antisense nucleotide is capable of binding to a nucleotide segment of the human MYH6 gene, as set forth in SEQ ID NO:2. In one embodiment, the antisense nucleotide is capable of binding the a nucleotide segment of the human MYH6 gene with sequence as set forth in SEQ ID NO:2 that has an G to A substitution in marker SG14S1131. Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention can be inhibited or blocked. In one embodiment, the antisense molecules are designed to specifically bind a particular allelic form of the target nucleic acid, thereby inhibiting translation of a product originating from this specific allele, but which do not bind other or alternate variants at the specific polymorphic sites of the target nucleic acid molecule. In one embodiment, the antisense molecule is designed to specifically bind to nucleic acids comprising the A allele of SG14S1131. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of the human MYH6 gene as set forth in SEQ ID NO:2, or a nucleotide sequence comprising, or consisting of, the complement of the nucleotide sequence of SEQ ID NO:2, wherein the nucleotide sequence comprises at least one polymorphic allele contained in the markers described herein (e.g., SG14S1131). The nucleic acid fragments of the invention are at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the invention is a system that is capable of carrying out a part or all of a method of the invention, or carrying out a variation of a method of the invention as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the invention includes one or more machines used for analysis of biological material (e.g., genetic material), as described herein. In some variations, this analysis of the biological material involves a chemical analysis and/or a nucleic acid amplification.

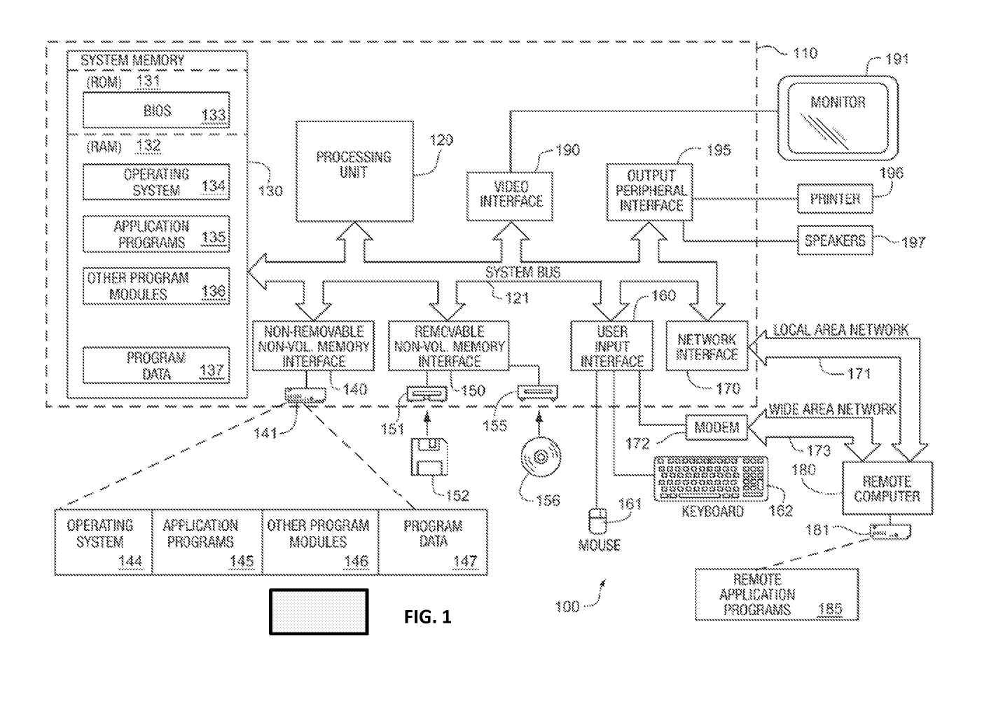

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

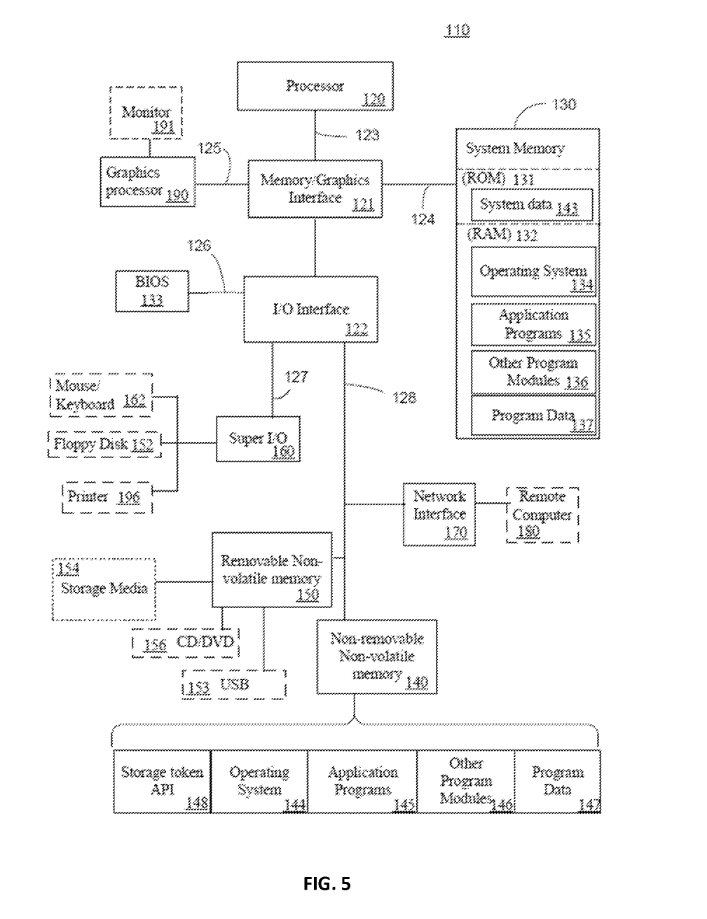

With reference to FIG. 5, a second exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 5. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (ISA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 5 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 5 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 1, provide storage of computer readable instructions, data structures, program modules and other data for the computer 110. In FIG. 5, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 5 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to a condition selected from Sick Sinus Syndrome, Atrial Fibrillation, Pacemaker implantation and Thoracic aortic aneurysm in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, a system of the invention is a system for identifying susceptibility to a cancer in a human subject, and comprises:
(a) at least one processor;
(b) at least one computer-readable medium;
(c) a susceptibility database operatively coupled to a computer-readable medium of the system and containing population information correlating the presence or absence of one or more alleles of the human MYH6 gene and susceptibility to a condition selected from Sick Sinus Syndrome, Atrial Fibrillation, Pacemaker implantation and Thoracic aortic aneurysm in a population of humans;
(d) a measurement tool that receives an input about the human subject and generates information from the input about the presence or absence of at least one mutant MYH6 allele in the human subject; and
(e) an analysis tool or routine that:
   (i) is operatively coupled to the susceptibility database and the information generated by the measurement tool,
   (ii) is stored on a computer-readable medium of the system,
   (iii) is adapted to be executed on a processor of the system, to compare the information about the human subject with the population information in the susceptibility database and generate a conclusion with respect to susceptibility to the condition for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

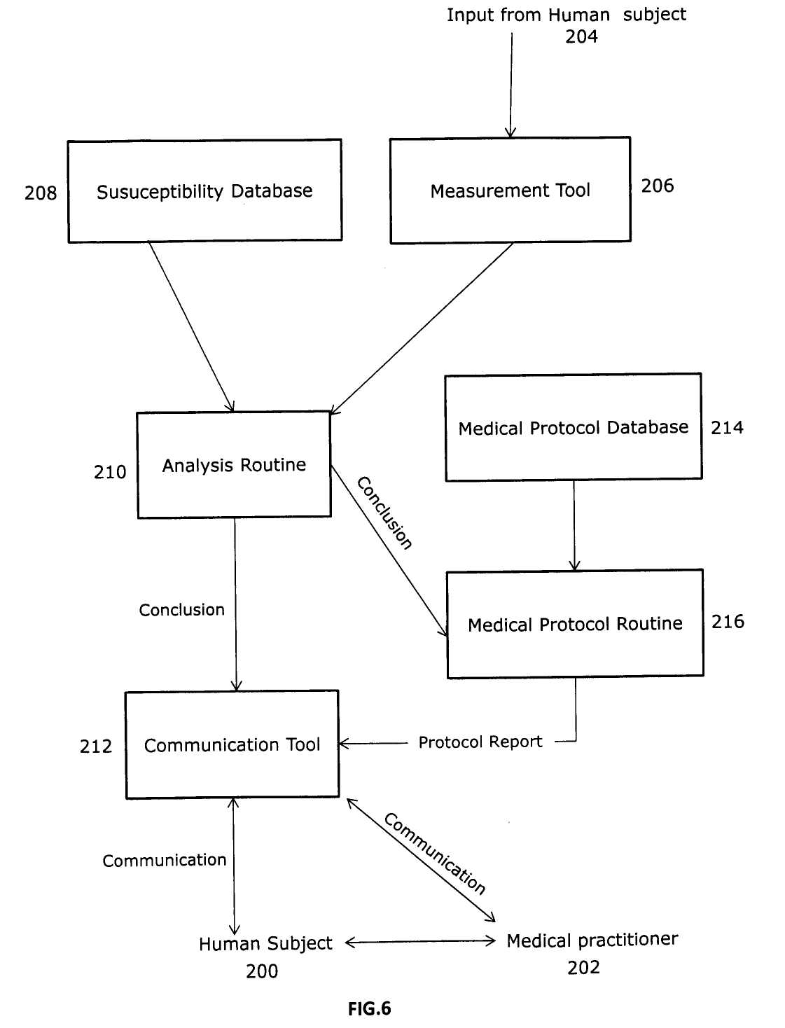

Referring to FIG. 6, an exemplary system includes a susceptibility database 208 that is operatively coupled to a computer-readable medium of the system and that contains population information correlating the presence or absence of one or more alleles of the human MYH6 gene and susceptibility to a condition selected from Sick Sinus Syndrome, Atrial Fibrillation, Pacemaker implantation and Thoracic aortic aneurysm in a population of humans. For example, the one or more alleles of the MYH6 gene include mutant alleles that cause, or are indicative of, a MYH6defect such as reduced or lost function, as described elsewhere herein.

In a simple variation, the susceptibility database contains 208 data relating to the frequency that a particular allele of MYH6 has been observed in a population of humans with the condition and a population of humans free of the condition. Such data provides an indication as to the relative risk or odds ratio of developing the condition for a human subject that is identified as having the allele in question. In another variation, the susceptibility database includes similar data with respect to two or more alleles of MYH6, thereby providing a useful reference if the human subject has any of the two or more alleles. In still another variation, the susceptibility database includes additional quantitative personal, medical, or genetic information about the individuals in the database diagnosed with the cancer or free of the cancer. Such information includes, but is not limited to, information about parameters such as age, sex, ethnicity, race, medical history, weight, diabetes status, blood pressure, family history of the condition, smoking history, and alcohol use in humans and impact of the at least one parameter on susceptibility to the condition. The information also can include information about other genetic risk factors for the condition besides MYH6 alleles. These more robust susceptibility databases can be used by an analysis routine 210 to calculate a combined score with respect to susceptibility or risk for developing the condition.

In addition to the susceptibility database 208, the system further includes a measurement tool 206 programmed to receive an input 204 from or about the human subject and generate an output that contains information about the presence or absence of the at least one MYH6 allele of interest. (The input 204 is not part of the system per se but is illustrated in the schematic FIG. 6.) Thus, the input 204 will contain a specimen or contain data from which the presence or absence of the at least one MYH6 allele can be directly read, or analytically determined. In a simple variation, the input contains annotated information about genotypes or allele counts for MYH6 in the genome of the human subject, in which case no further processing by the measurement tool 206 is required, except possibly transformation of the relevant information about the presence/absence of the MYH6 allele into a format compatible for use by the analysis routine 210 of the system.

In another variation, the input 204 from the human subject contains data that is unannotated or insufficiently annotated with respect to MYH6, requiring analysis by the measurement tool 206. For example, the input can be genetic sequence of a chromosomal region or chromosome on which MYH6 resides, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one mutant MYH6 allele in a human subject from the data. For example, the measurement tool 206 contains instructions, preferably executable on a processor of the system, for analyzing the unannotated input data and determining the presence or absence of the MYH6 allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant MYH6 allele from the genomic sequence information.

In yet another variation, the input 204 from the human subject comprises a biological sample, such as a fluid (e.g., blood) or tissue sample that contains genetic material that can be analyzed to determine the presence or absence of the MYH6 allele of interest. In this variation, an exemplary measurement tool 206 includes laboratory equipment for processing and analyzing the sample to determine the presence or absence (or identity) of the MYH6 allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one MYH6 allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one mutant MYH6 allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to the condition for the human subject. In simple terms, the analysis tool 210 looks at the MYH6 alleles identified by the measurement tool 206 for the human subject, and compares this information to the susceptibility database 208, to determine a susceptibility to the condition for the subject. The susceptibility can be based on the single parameter (the identity of one or more MYH6 alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to the condition. Such a conclusion is expressed in the conclusion in any statistically useful form, for example, as an odds ratio, a relative risk, or a lifetime risk for subject developing the condition.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 6, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to the condition for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to a cancer when a medical practitioner can prescribe or administer a standard of care that can reduce susceptibility to the condition; and/or delay onset of the condition; and/or increase the likelihood of detecting the condition at an early stage. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery, including in extreme cases pacemaker placement.

For example, in some variations, the system further includes a medical protocol database 214 operatively connected to a computer-readable medium of the system and containing information correlating the presence or absence of the at least one MYH6 allele of interest and medical protocols for human subjects at risk for the condition. Such medical protocols include any variety of medicines, lifestyle changes, diagnostic tests, increased frequencies of diagnostic tests, and the like that are designed to achieve one of the aforementioned goals. The information correlating a MYH6 allele with protocols could include, for example, information about the success with which the condition is avoided or delayed, or success with which the condition is detected early and treated, if a subject has a MYH6 susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to condition for the subject) and the medical protocol database 214, and (ii) generate a protocol report with respect to the probability that one or more medical protocols in the medical protocol database will achieve one or more of the goals of reducing susceptibility to the condition; delaying onset of the condition; and increasing the likelihood of detecting the condition at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system include the communication tool 212. In some examples, the communication tool generates a communication that includes the protocol report in addition to, or instead of, the conclusion with respect to susceptibility.

Information about MYH6 allele status not only can provide useful information about identifying or quantifying susceptibility to the condition; it can also provide useful information about possible causative factors for a human subject identified with the condition, and useful information about therapies for the patient. In some variations, systems of the invention are useful for these purposes.

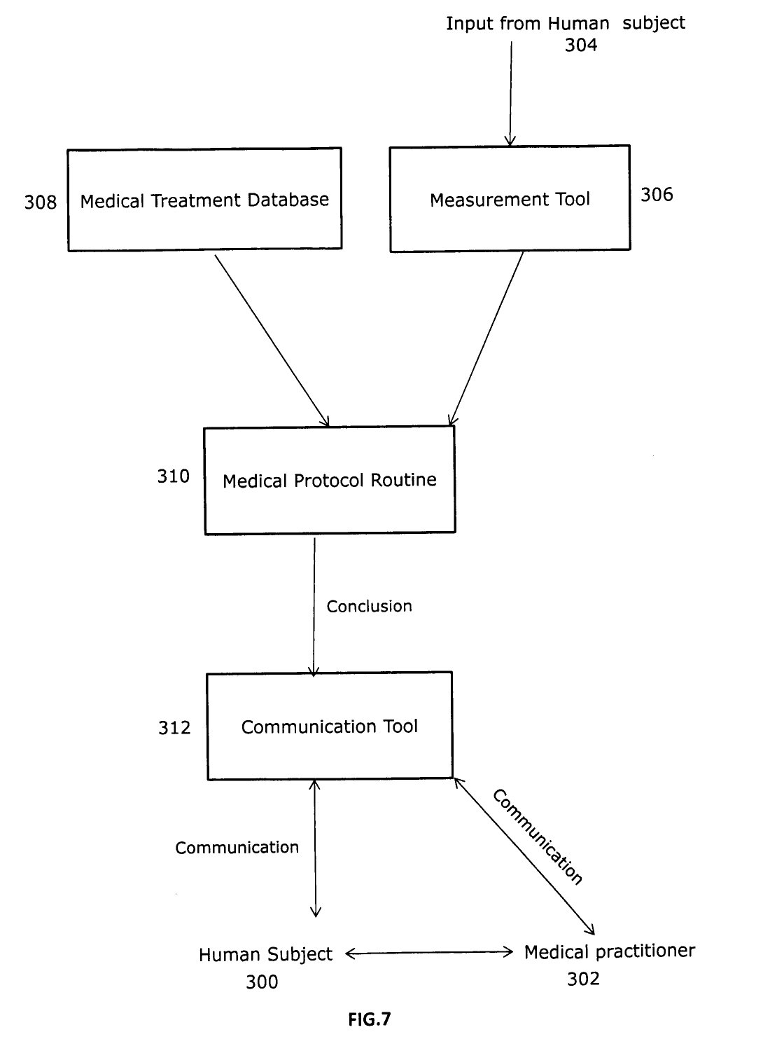

For instance, in some variations the invention is a system for assessing or selecting a treatment protocol for a subject diagnosed with a a condition selected from Sick Sinus Syndrome, Atrial Fibrillation, Pacemaker implantation and Thoracic aortic aneurysm. An exemplary system, schematically depicted in FIG. 7, comprises:
(a) at least one processor;
(b) at least one computer-readable medium;
(c) a medical treatment database 308 operatively connected to a computer-readable medium of the system and containing information correlating the presence or absence of at least one MYH6 allele and efficacy of treatment regimens for the condition;
(d) a measurement tool 306 to receive an input (304, depicted in FIG. 7 but not part of the system per se) about the human subject and generate information from the input 304 about the presence or absence of the at least one MYH6 allele in a human subject diagnosed with the condition; and
(e) a medical protocol routine or tool 310 operatively coupled to the medical treatment database 308 and the measurement tool 306, stored on a computer-readable medium of the system, and adapted to be executed on a processor of the system, to compare the information with respect to presence or absence of the at least one MYH6 allele for the subject and the medical treatment database, and generate a conclusion with respect to at least one of:
   (i) the probability that one or more medical treatments will be efficacious for treatment of the condition for the patient; and
   (ii) which of two or more medical treatments for the condition will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 7, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In a further embodiment, the invention provides a computer-readable medium having computer executable instructions for determining susceptibility to Sick Sinus Syndrome in a human individual, the computer readable medium comprising (i) sequence data identifying at least one allele of at least one polymorphic marker in the individual; and (ii) a routine stored on the computer readable medium and adapted to be executed by a processor to determine risk of developing Sick Sinus Syndrome for the at least one polymorphic marker; wherein the at least one polymorphic marker is a marker in the human MYH6 gene, or an amino acid substitution in an encoded MYH6 protein, that is predictive of susceptibility of Sick Sinus Syndrome in humans. In one embodiment, the at least one polymorphic marker is SG14S1131. In another embodiment, the amino acid substitution is the R721W substitution.

In certain embodiments, a report is prepared, which contains results of a determination of susceptibility of the condition. The report may suitably be written in any computer readable medium, printed on paper, or displayed on a visual display.

The present invention will now be exemplified by the following non-limiting examples.

### Example 1

With the aim to search for sequence variants that predispose to SSS, we performed a genome-wide association study (GWAS) on 792 Icelandic SSS cases and 37,592 Icelandic population controls (Table 1). All cases had received the SSS diagnosis at Landspitali University Hospital (LUH) in Reykjavik, Iceland, the only tertiary hospital in Iceland. Of 792 SSS cases, 627 had undergone a Pacemaker (PM) implantation.

Initially, we tested a total of 7.2 million single nucleotide polymorphisms (SNPs) either directly genotyped with the Illumina HumanHap300/CNV370 chips or imputed from one or more of four sources, the HapMap2 CEU sample¹⁰ (60 triads), the 1000 Genomes data¹¹ (179 individuals) and Icelandic samples genotyped with the Illumina Human1M-Duo (123 triads) and the HumanOmni1-Quad chips (505 individuals). Imputations were based on the IMPUTE model¹² and long range phasing of chip typed Icelandic samples¹³.

The association analysis yielded genome-wide significant association (P<5×10⁻⁸) between SSS and three correlated SNPs at chromosome 14q11: rs2231801 (imputed from the Human1M-Duo and HumanOmni1-Quad chips), rs28730774 (imputed from the HumanOmni1-Quad chip) and 14-22399934 (imputed from the 1000 Genomes project) (most significant P=1.3×10⁻¹³ for rs2231801). For all three SNPs the imputed minor allele frequency was low (0.010-0.026) and the minor allele was the at risk allele.

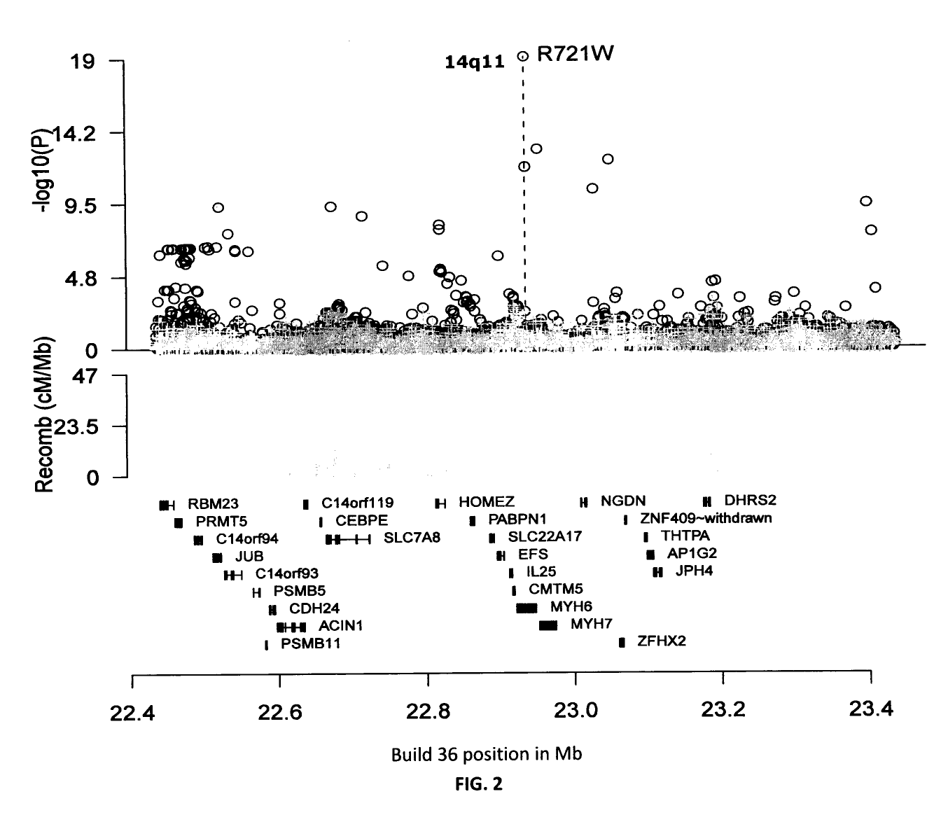

In an effort to search for additional signals associating with SSS and to explore the observed association at 14q11, we selected seven chip typed SSS cases for whole-genome sequencing, four of whom carry the rs28730774[T] variant (FIG. 2). Another 80 chip typed Icelanders, not diagnosed with SSS, were also whole-genome sequenced, for a total of 87 samples that were sequenced to a mean mapped sequencing depth of ten-fold. Based on this sequencing data, a total of 11 million SNPs were called and imputed into chip typed cases and controls, applying the long-range phased haplotypes, and tested for association with SSS. No significant association was found outside the 14q11 region. In this region, the strongest association observed was with a single nucleotide substitution (SG14S1131) in MYH6, the gene encoding the alpha heavy chain subunit of cardiac myosin (G->A substitution). This substitution encodes a missense mutation, R721W, in MYH6 (FIGS. 2, 3 and 4). This mutation was present in all four SSS rs28730774[T] carrier cases but absent from both the non-carriers and a single control carrier of rs28730774[T]. The substitution encoding R721W is located in exon 18 of MYH6 at position 22,936,019 (in Build 36 Assembly of the UCSC Genome Browser) and results in a missense R->W mutation at amino acid 721. No significant association remained at 14q11 after accounting for the effect of R721W (FIG. 2).

In order to validate the R721W mutation we used Sanger sequencing to resequence exon 18 of MYH6 in a set of 351 Icelanders enriched for carriers of rs28730774[T]. We then genotyped SG14S1131 encoding the R721W mutation directly with a Centaurus single SNP assay in the same sample set and the two genotyping methods concurred for all 351 individuals. In this set, 82 individuals were called as heterozygous carriers of the R721W mutation and none was called a homozygous carrier. We subsequently genotyped additional 523 chip typed Icelanders with the Centaurus assay, four of whom were called heterozygous carriers for R721W. Amongst the two sample sets combined there were 130 carriers of rs28730774[T]. The R721W mutation was only observed amongst carriers of rs28730774[T] and was found in 91% of SSS case carriers but only in 38% of control carriers. This presents a clear refinement of the association with SSS (P=2.9×10⁻⁹, Table 3).

The 87 whole-genome sequenced samples and the 874 Centaurus single SNP assay and chip genotyped individuals were combined into a set of 952 individuals (five individuals overlapped and nine could not be phased accurately in the region around MYH6), of which 73 are carriers of the R721W mutation, to create a new reference set for imputation. Based on this imputation set, the association is even stronger than when based on the initial smaller imputation set. The R721W missense mutation in MYH6 (estimated allelic frequency in Iceland=0.0038) associates with SSS with an OR of 12.53 (95% CI: 8.08, 19.44) and a corresponding P value of 1.5×10⁻²⁹. A second set of 449 Icelandic SSS cases who were not chip genotyped, and therefore unavailable for the haplotype based imputation, were directly genotyped for the R721W mutation with a Centaurus single SNP assay. In this set, 21 heterozygous carriers of R721W were identified (allelic frequency of 0.023), which replicates the original association with SSS based on imputations where the estimated allelic frequencies in SSS cases and controls were 0.031 and 0.0038, respectively.

The sibling recurrence risk ratio (λ_{sibling}) is a commonly used measure of the strength of familial aggregation. Based on allelic frequency of R721W of 0.0038 and an OR for SSS of 12.53 we estimate the λ_{sibling} for SSS accounted for by R721W to be 1.52. This contrasts most of the sequence variants that in recent years have been found to associate with common complex disorders, that create λ_{sibling} that is substantially less than 1.1. Despite the high OR, the R721W mutation is also distinct from most Mendelian disorder mutations by its higher population frequency. There is, however, a striking similarity with the Icelandic 999del5 BRCA2 breast cancer mutation¹⁴ that is almost exclusive to Iceland and we estimate to have an allelic frequency of 0.0043 (based on 20,635 males) and an OR of 11.4 (based on 2,466 female cases and 25,357 female controls) in Iceland, leading to a λ_{sibling} of 1.47.

The 73 chip typed individuals who were shown to carry the R721W mutation through direct genotyping all share a 26 SNP haplotype spanning 230 kb or 0.51 centi-Morgans. Using surrounding SNPs we estimate the age of the R721W mutation to be 29 generations or approximately 270 years^{15,16}.

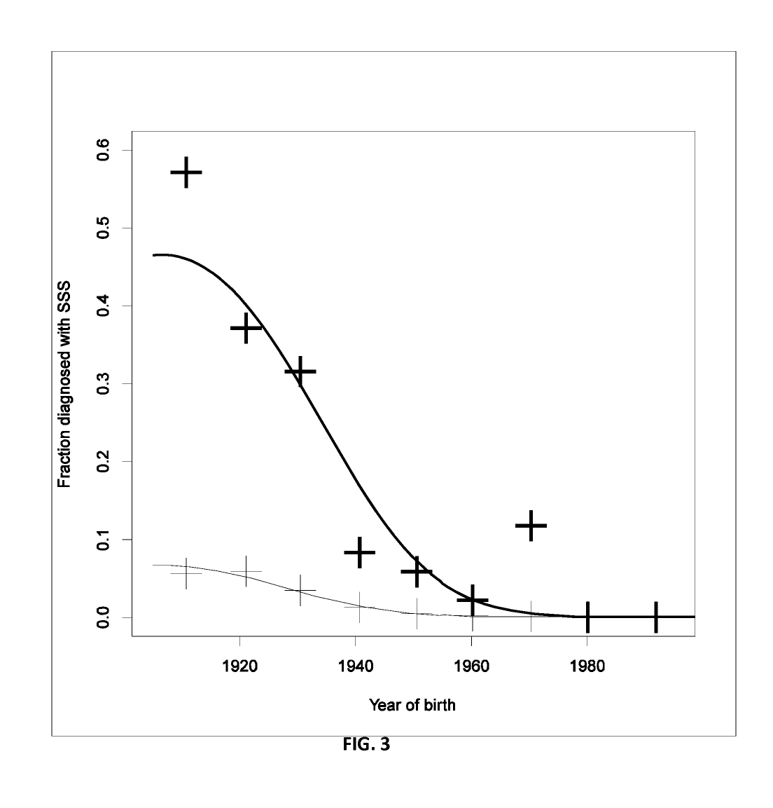

Based on data from the 38,384 chip typed Icelanders, including both SSS cases and controls, the lifetime risk of being diagnosed with SSS is ˜6% for non-carriers of R721W but ˜50% for carriers of the R721W mutation (FIG. 3). Notably, the chip typed Icelanders were not selected for genotyping based on SSS or any other phenotypes related to SSS and can therefore be used to assess the probability of developing SSS depending on genotype and age. The effect of R721W as estimated by our logistic model showed a trend towards increasing with age by a factor of 1.06 per ten years (P=0.042).

To explore the relationship between SSS, genotype and syncope, a common but non-specific sign of SSS, we obtained a list of individuals who had received the discharge diagnosis of syncope (syncope, convulsions or dizziness, see Supplementary Methods) at the LUH between the years 1987 and 2010, either through a visit to the emergency room or hospital admission. For our analysis we used data from the 916 patients who had previously been chip typed at deCODE (mean year of birth=1929.7). We observed that amongst patients with syncope, 50% of carriers of the R721W mutation had been diagnosed with SSS, compared to 19% of the non-carriers.

Given the fact that SSS is commonly associated with other cardiac disease processes, particularly other conduction disorders, we tested for association between the R721W mutation and several other cardiovascular diseases (Table 4). We analysed Icelandic sample sets both before and after exclusion of known cases of SSS. Although these results can only be considered as suggestive, it is interesting to observe residual association, after exclusion of SSS cases, with several diseases, including atrial fibrillation and thoracic aortic aneurysm.

Cardiac muscle myosin, along with actin, is one of the major components of the sarcomere, the building block of the contractile system of cardiac muscle¹⁷. Myosin is a hexamer consisting of two heavy chain subunits, two light chain subunits and two regulatory subunits. MYH6 encodes the alpha heavy chain subunit (αMHC), one of two sarcomeric MHC isoforms that are expressed in the mammalian myocardium, encompasses ˜26K base pairs and consists of 39 exons, 37 of which contain coding information¹⁸. βMHC, a relatively slow adenosine triphosphatase (ATPase), encoded by the MYH7 gene, is the heavy chain isoform predominantly expressed in human hearts, with expression of αMHC, a fast ATPase, primarily restricted to atrial tissue¹⁹. The two genes are oriented in a head-to-tail tandem on chromosome 14²⁰ and are regulated in an antithecal manner. In heart failure and other cardiac disorders in humans, βMHC is upregulated while αMHC is downregulated, resulting in diminution of cardiac performance²¹, and it has been suggested that even minor shifts in MHC composition of the cardiac muscle can markedly influence cardiac function. Mutations in MYH7 are a well-known cause of hypertrophic cardiomyopathy in man, and recently, mutations in MYH6 were also linked to both cardiomyopathy (hypertrophic and dilated)²² and a variety of congenital heart defects²³.

While neither MYH6 nor MYH7 have been associated with SSS or other arrhythmias in man before, we have recently established a link between a common variant in MYH6 and cardiac conduction. Through a large GWAS on ECG measures in Icelanders²⁴, we discovered an association between a missense variant in exon 25 of MYH6 (rs365990[G], A1101V) and both heart rate and the PR interval. The G allele of rs365990 (frequency=0.341) associates with a 0.91 beat decrease in heart rate per minute. The association between rs365990[G] and heart rate has since been replicated in a large meta-analysis including subjects of European ancestry from both the US and Europe²⁵. There is, however, no significant association between this variant and SSS in our data. The R721W mutation occurs on the background of the G allele of rs365990 and when tested in our previously described ECG database²⁴, after exclusion of all cases with SSS and PM, was found to associate with a 5.05 beat decrease in heart rate per minute (P=0.003) and prolongation of the PR interval (Table 2). The association of R721W with lower heart rate in subjects that have not been diagnosed with SSS, indicates a higher penetration of this mutation than suggested by purely assessing the association with SSS and need for PM placement, but with variability in expression resulting in phenotypic variability.

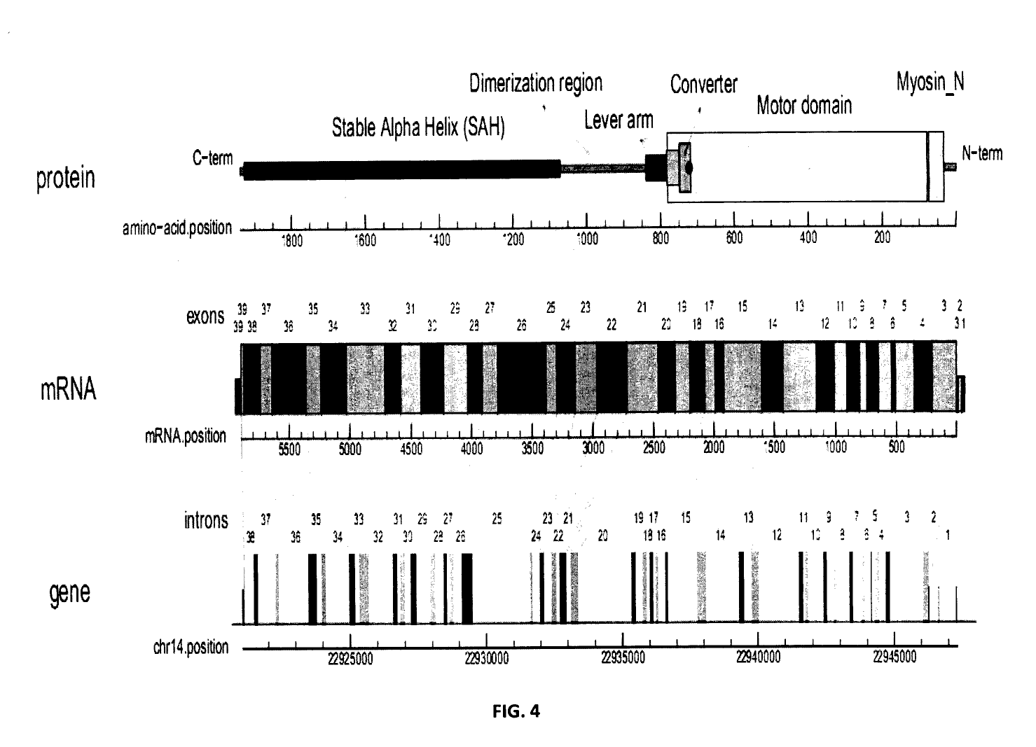

The R721W mutation is in exon 18 that encodes for part of the converter domain of αMHC (FIG. 4). This domain functions as a socket for the C-terminal α-helical tail of the αMHC and plays a critical role in amplifying the structural rearrangements in the motor domain and transmitting them to the α-helical tail during movements of the myosin during contraction²⁶. Based on PolyPhen²⁷ the R721W mutation is predicted to alter the structure of the converter. How this structural alteration can directly affect cardiac conduction is pending further structure-function analysis of the protein. Although the αMHC protein itself has not been directly linked to cardiac conduction, a cardiac-specific highly conserved microRNA, miR-208a, encoded by intron 27 of Myh6 in both humans and mice, has recently been shown in mice to be necessary for maintenance of normal cardiac conduction²⁷. Both gain and loss of miR-208a function were associated with conduction abnormalities including prolonged PR (first-degree atrioventricular block), second-degree atrioventricular block and atrial fibrillation. No effect on heart rate was reported. The miR-208a is also an important regulator of cardiac gene expression in response to stress^{28,29}. Evidence suggests that miR-208a is required for expression of Cx40³⁰, of particular interest as mice lacking Cx40 exhibit cardiac conduction abnormalities, including of both sinus node impulse formation and atrial propagation^{31 32 33}. A potential functional consequence of the R721W mutation could be an effect on mRNA stability or processing, as previously documented for both synonymous and nonsynonymous coding mutations³³⁻³⁵ and thus directly affecting the amount of miR-208a present in the heart.

### Methods

The study was approved by the Data Protection Commission of Iceland and the National Bioethics Committee of Iceland. Written informed consent was obtained from all study participants. Personal identifiers associated with medical information and blood samples were encrypted with a third-party encryption system as provided by the Data Protection Commission of Iceland.

### Icelandic Sample Sets.

The sick sinus syndrome (SSS) sample set included patients who received the discharge diagnosis of SSS (ICD 10 code 149.5, ICD 9 code 427.8) at Landspitali University Hospital (LUH) in Reykjavik between 1987 and 2010. All diagnoses were confirmed with a 12-lead ECG. The pacemaker (PM) population sample set included all patients who received a PM implantation at the LUH between 1987 and 2010. The causes of PM implantation among the 930 cases with genotype information break down as follows: SSS only, N=466; AVB only, N=189; AF, N=147; other cause, N=63; SSS and AF, N=19; SSS and AVB, N=12; AF and AVB, N=2; no known cause, N=32. All diagnoses were confirmed with a 12-lead ECG. The syncope sample set consisted of individuals that received the diagnosis of syncope, convulsion or dizziness (ICD 10 codes R55 and R56, ICD 9 codes 780.2, 780.3, 780.39 and 780.4) at the LUH between the years 1987 and 2010, either through a visit to the emergency room or hospital admission. Other samples have been described elsewhere^{24,37}. The controls used in the various case-control analyses of this study consisted of disease-free controls randomly drawn from the Icelandic genealogical database and individuals from other ongoing genetic studies at deCODE. Potential controls with first-degree relatives with the relevant disease and those with a first-degree control relative were excluded from all analyses.

### Illumina Genome-Wide Genotyping.

The Icelandic chip typed samples were assayed with the Illumina HumanHap300 or HumanHap CNV370 bead chips at deCODE genetics. The bead chips contained 317,503 and 370,404 haplotype tagging SNPs derived from phase I of the International HapMap project. Only SNPs present on both chips were included in the analysis and SNPs were excluded if they had (i) yield lower than 95% in cases or controls, (ii) minor allele frequency less than 1% in the population, or (iii) showed significant deviation from Hardy-Weinberg equilibrium in the controls (P<0.001). All samples with a call rate below 98% were excluded from the analysis. The final analysis was based on direct genotyping of 289,658 autosomal SNPs.

### Single SNP Genotyping.

Single SNP genotyping was carried out by deCODE genetics applying the Centaurus (Nanogen) platform³⁸. The quality of each Centaurus SNP assay was evaluated by genotyping each assay on the CEU samples and comparing the results with the HapMap data. All assays had mismatch rate <0.5%. Additionally, all markers were genotyped again on more than 10% of samples typed with the Illumina platform, resulting in an observed mismatch in less than 0.5% of samples.

### Sanger Sequencing.

Dye-terminator Sanger sequencing was performed with the Applied Biosystems BigDye® Terminator v3.1 Cycle Sequencing Kit alongside with Agencourt® Ampure® XP and Agencourt® CleanSeq® for cleanup of the PCR and cycle sequencing product, respectively. Ampure®XP and CleanSeq® beads cleaning was performed on Zymark® Sciclone ALH-500 liquid handling robot system. Tray dilutions for PCR setup and cycle sequencing setup were prepared on Packard MultiprobeII® HTEX liquid handling robot system and genomic DNA and PCR product were transferred into the respective trays using Zymark® Sciclone ALH-500. PCR and cycle sequencing reactions were performed on MJ Research PTC-225 thermal cyclers. For signal detection Applied Biosystems 3730×1 DNA analyzers were used.

### Whole-Genome Sequencing.

Sample preparation: Paired-end libraries for sequencing were prepared according to manufacturer's instructions (Illumina). In short, approximately 5 micrograms of genomic DNA, isolated from frozen blood samples, was fragmented to a mean target size of 300 basepairs (bp) using a Covaris E210 instrument. The resulting fragmented DNA was end-repaired using T4 and Klenow polymerases and T4 polynucleotide kinase with 10 mM dNTP's followed by addition of an “A” base at the ends using Klenow exo fragment (3′ to 5′-exo minus) and dATP (1 mM). Sequencing adaptors containing “T” overhangs were ligated to the DNA products followed by agarose (2%) gel electrophoresis. Fragments of about 400 bp were isolated from the gels (Qiagen Gel Extraction Kit) and the adaptor-modified DNA fragments were PCR enriched for 10-cycles using Phusion DNA polymerase (Finnzymes Oy) and PCR primers PE 1.0 and PE 2.0 (Illumina). Enriched libraries were further purified using agarose (2%) gel electrophoresis as described above. The quality and concentration of the libraries was assessed with the Agilent 2100 Bioanalyzer using the DNA 1000 LabChip (Agilent). Barcoded libraries were stored at −20° C. All steps in the workflow were monitored using an in-house laboratory information management system with barcode tracking of all samples and reagents. DNA sequencing: Template DNA fragments were hybridized to the surface of flow cells (Illumina PE flowcell, v4) and amplified to form clusters using the Illumina cBot. In brief, DNA (8-10 pM) was denatured followed by hybridization to grafted adaptors on the flowcell. Isothermal bridge amplification using Phusion polymerase was then followed by linearization of the bridged DNA, denaturation, blocking of 3′-ends and hybridization of the sequencing primer. Sequencing-by-synthesis was performed on Illumina GAIIx instruments equipped with paired-end modules. Paired-end libraries were sequenced using 2×101 cycles of incorporation and imaging with Illumina sequencing kits, v4. Each library/sample was initially run on a single lane for validation followed by further sequencing of ≧4 lanes with targeted cluster densities of 250-300K/mm². Imaging and analysis of the data was performed using the SCS 2.6 and RTA 1.6 software packages from Illumina, respectively. RTA analysis involved conversion of image data to base-calling in real-time. Alignment: For each lane in the DNA sequencing output, the resulting qseq files were converted into fastq files using an inhouse script. All output from sequencing was converted and the Illumina quality filtering flag was retained in the output. The fastq files were then aligned against Build 36 of the human reference sequence using bwa version 0.5.7³⁹. BAM file generation: SAM file output from the alignment was converted into BAM format using samtools version 0.1.8⁴° and an inhouse script was used to carry the Illumina quality filter flag over to the BAM file. The BAM files for each sample were then merged into a single BAM file using samtools. Finally, Picard version 1.17 (see http://picard.sourceforge.net/) was used to mark duplicates in the resulting sample BAM files.

### SNP Calling and Genotyping in Whole-Genome Sequencing.

A two step approach was applied to SNP genotyping the whole-genome sequencing data. First, a SNP detection step where sequence positions where at least one individual could be determined to be different from the reference sequence with confidence (quality threshold of 20) based on the SNP calling feature of the pileup tool of samtools⁴⁰. SNPs that were always heterozygous, or always homozygous different from the reference were removed. Second, all positions that were flagged as polymorphic were then genotyped using the pileup tool, but since sequencing depth varies and hence the certainty of genotype calls, genotype likelihoods were calculated rather than deterministic calls.

### Long Range Phasing.

Long ranged phasing of all chip genotyped individuals was performed with methods described previously^{13,41}. In brief, phasing is achieved using an iterative algorithm which phases a single proband at a time, given the available phasing information about everyone else that shares a long haplotype identically by state with the proband. Given the large fraction of the Icelandic population that has been chip typed accurate long range phasing is available genome-wide for all chip typed Icelanders.

### Genotype Imputation.

We impute the SNPs identified and genotyped through sequencing into all Icelanders that have been phased using long range phasing using the model used by IMPUTE¹². The genotype data from sequencing can be ambiguous due to low sequencing coverage and is not phased. In order phase the sequencing genotypes an iterative algorithm was applied for each SNP with alleles 0 and 1. Let H be the long range phased haplotypes of the sequenced individuals and follow:
1. For each haplotype h in H, use the hidden Markov model of IMPUTE to calculate γ_{h,k} for every other k in H, a measure of how likely h is to have the same ancestral source as k.
2. For every h in H initialize the parameter θₕ which specifies how likely the 1 allele of the SNP is to occur on the background of h from the genotype likelihoods obtained from sequencing. If L₀, L₁ and L₂ are the likelihoods of the genotypes 0, 1 and 2 in the individual that carries h, then set

$${θ}_{h} = \frac{{L}_{2} + \frac{1}{2} {L}_{1}}{{L}_{2} + {L}_{1} + {L}_{0}} .$$

3. For every pair of haplotypes h and k in H that are carried by the same individual use the other haplotypes in H to predict the genotype of the SNP on the backgrounds of h and k:

$${τ}_{h} = {\sum }_{l ∈ H \backslash \{ h \}} {γ}_{h , l} {θ}_{l}$$

$$and$$

$${τ}_{k} = {\sum }_{l ∈ H \backslash \{ k \}} {γ}_{k , l} {θ}_{l} .$$

Combining these predictions with the genotype likelihoods from sequencing gives un-normalized updated phased genotype probabilities: P₀₀=(1−τₕ)(1−τₖ)L₀, P₀₁=(1−τₕ)τₖ½L₁, P₁₀=τTₕ(1−τₖ)½L₁ and P₁₁=τₕτₖL₂. Now use these values to update θₕ and θₖ to

$$\frac{{P}_{10} + {P}_{11}}{{P}_{00} + {P}_{01} + {P}_{10} + {P}_{11}}$$

$$and$$

$$\frac{{P}_{01} + {P}_{11}}{{P}_{00} + {P}_{01} + {P}_{10} + {P}_{11}} ,$$

respectively.
4. Repeat step 3 while the maximum difference between iterations is greater than ε. We used ε=10⁻⁷.

Given the long range phased haplotypes and θ the allele of the SNP on a new haplotype h, not in H, is imputed as

$${\sum }_{l ∈ H} {γ}_{h , l} {θ}_{l} .$$

The above algorithm can easily be extended to handle simple family structures such as parent offspring pairs and triads by letting the P distribution run over all founder haplotypes in the family structure. The algorithm also extends trivially to the X-chromosome. If source genotype data is only ambiguous in phase, such as chip genotype data, then the algorithm is still applied but all but one of the Ls will be 0.

### Association Testing.

Logistic regression was used to test for association between SNPs and disease, treating disease status as the response and expected genotype counts from imputation or allele counts from direct genotyping as covariates. Testing was performed using the likelihood ratio statistic. No additional covariates were used in the genome-wide association scan, but in additional investigation of R721W, year of birth, and year of birth squared were also used as covariates. The conditional analysis of the region around R721W was performed by adding the R721W as a covariate while testing every SNP in the region for association with SSS. Similarly, association with the quantitative ECG parameters was performed using linear regression, treating the ECG measurement as the response and the genotype as the covariate. Conditional analysis was performed by adding the allele count of the SNP being conditioned on as a covariate.

### REFERENCES

1. Ferrer, M. I. The sick sinus syndrome in atrial disease. Jama 206, 645-6 (1968).
2. Saksena, S., Camm J. A. Electrophysiological Disorders of the Heart, (Elsevier Churchill Livingstone, Philadelphia, 2004).
3. Epstein, A. E. et al. ACC/AHA/HRS 2008 Guidelines for Device-Based Therapy of Cardiac Rhythm Abnormalities: a report of the American College of Cardiology/American Heart Association Task Force on Practice Guidelines (Writing Committee to Revise the ACC/AHA/NASPE 2002 Guideline Update for Implantation of Cardiac Pacemakers and Antiarrhythmia Devices) developed in collaboration with the American Association for Thoracic Surgery and Society of Thoracic Surgeons. J Am Coll Cardiol 51, e1-62 (2008).
4. Kusumoto, F. M. & Goldschlager, N. Cardiac pacing. N Engl J Med 334, 89-97 (1996).
5. Benson, D. W. et al. Congenital sick sinus syndrome caused by recessive mutations in the cardiac sodium channel gene (SCN5A). J Clin Invest 112, 1019-28 (2003).
6. Schulze-Bahr, E., M. W. et al. PacemakerContribution of sodium channel mutations to bradycardia and sinus node dysfunction in a patient with sinus node diseaseLQT3 families. J Clin InvestCirc Res 11192, 1537-45976-83 (2003).
7. Milanesi, R., Baruscotti, M., Gnecchi-Ruscone, T. & DiFrancesco, D. Familial sinus bradycardia associated with a mutation in the cardiac pacemaker channel. N Engl J Med 354, 151-7 (2006).
8. Mohler, P. J. et al. Ankyrin-B mutation causes type 4 long-QT cardiac arrhythmia and sudden cardiac death. Nature 421, 634-9 (2003).
9. Dobrzynski, H., Boyett, M. R. & Anderson, R. H. New insights into pacemaker activity: promoting understanding of sick sinus syndrome. Circulation 115, 1921-32 (2007).
10. A haplotype map of the human genome. Nature 437, 1299-320 (2005).
11. Durbin, R. M. et al. A map of human genome variation from population-scale sequencing. Nature 467, 1061-73.
12. Marchini, J., Howie, B., Myers, S., McVean, G. & Donnelly, P. A new multipoint method for genome-wide association studies by imputation of genotypes. Nat Genet 39, 906-13 (2007).
13. Kong, A. et al. Detection of sharing by descent, long-range phasing and haplotype imputation. Nat Genet (2008).
14. Thorlacius, S. et al. A single BRCA2 mutation in male and female breast cancer families from Iceland with varied cancer phenotypes. Nat Genet 13, 117-9 (1996).
15. Slatkin, M. & Rannala, B. Estimating allele age. Annu Rev Genomics Hum Genet 1, 225-49 (2000).
16. Labuda, M. et al. Linkage disequilibrium analysis in young populations: pseudo-vitamin D-deficiency rickets and the founder effect in French Canadians. Am J Hum Genet 59, 633-43 (1996).
17. Fuster, V. (ed.) Hurst's The Heart, (McGraw Hill, 2004).
18. Epp, T. A., Dixon, I. M., Wang, H. Y., Sole, M. J. & Liew, C. C. Structural organization of the human cardiac alpha-myosin heavy chain gene (MYH6). Genomics 18, 505-9 (1993).
19. Franco, D., Lamers, W. H. & Moorman, A. F. Patterns of expression in the developing myocardium: towards a morphologically integrated transcriptional model. Cardiovasc Res 38, 25-53 (1998).
20. Mandavi, V., Chambers, A. P. & Nadal-Ginard, B. Cardiac alpha- and beta-myosin heavy chain genes are organized in tandem. Proc Natl Acad Sci USA 81, 2626-30 (1984).
21. Miyata, S., Minobe, W., Bristow, M. R. & Leinwand, L. A. Myosin heavy chain isoform expression in the failing and nonfailing human heart. Circ Res 86, 386-90 (2000).
22. Carniel, E. et al. Alpha-myosin heavy chain: a sarcomeric gene associated with dilated and hypertrophic phenotypes of cardiomyopathy. Circulation 112, 54-9 (2005).
23. Granados-Riveron, J. T. et al. {alpha}-Cardiac myosin heavy chain (MYH6) mutations affecting myofibril formation are associated with congenital heart defects. Hum Mol Genet.
24. Holm, H. et al. Several common variants modulate heart rate, PR interval and QRS duration. Nat Genet 42, 117-22.
25. Eijgelsheim, M. et al. Genome-wide association analysis identifies multiple loci related to resting heart rate. Hum Mol Genet 19, 3885-94.
26. Sweeney, H. L. & Houdusse, A. Myosin VI rewrites the rules for myosin motors. Cell 141, 573-82
27. Sunyaev, S. et al. Prediction of deleterious human alleles. Hum Mol Genet 10, 591-7 (2001).
28. van Rooij, E. et al. Control of stress-dependent cardiac hypertrophy and conduction in mice. Science 316, 575-9 (2007).
29. Callis, T. E. et al. MicroRNA-208a is a regulator of cardiac hypertrophy and conduction in mice. J Clin Invest 119, 2772-86 (2009).
30. Simon, A. M., Goodenough, D. A. & Paul, D. L. Mice lacking connexin40 have cardiac conduction abnormalities characteristic of atrioventricular block and bundle branch block. Curr Biol 8, 295-8 (1998).
31. Simon, A. M., Sutherland, L. B. & Paul, D. L. Control of stress-dependent cardiac growth and bundle branch block. Science 8, 575-9 (2007).
32. van Rooij, E. et al. A family of microRNAs encoded by myosin genes governs myosin expression and muscle performance. Dev Cell 17, 662-73 (2009).
33. Duan, J. et al. Synonymous mutations in the human dopamine receptor D2 (DRD2) affect mRNA stability and synthesis of the receptor. Hum Mol Genet 12, 205-16 (2003).
34. Wang, D., Johnson, A. D., Papp, A. C., Kroetz, D. L. & Sadee, W. Multidrug resistance polypeptide 1 (MDR1, ABCB1) variant 3435C>T affects mRNA stability. Pharmacogenet Genomics 15, 693-704 (2005).
35. Zhang, H. et al. Association between two mu-opioid receptor gene (OPRM1) haplotype blocks and drug or alcohol dependence. Hum Mol Genet 15, 807-19 (2006).
36. Johansen, C. T. et al. Excess of rare variants in genes identified by genome-wide association study of hypertriglyceridemia. Nat Genet. 42, 684-7.
37. Preuss, M. et al. Design of the Coronary ARtery DIsease Genome-Wide Replication And Meta-Analysis (CARDIoGRAM) Study: A Genome-wide association meta-analysis involving more than 22 000 cases and 60 000 controls. Circ Cardiovasc Genet 3, 475-83.
38. Kutyavin, I. V. et al. A novel endonuclease IV post-PCR genotyping system. Nucleic Acids Res 34, e128 (2006).
39. Li, H. & Durbin, R. Fast and accurate short read alignment with Burrows-Wheeler transform. Bioinformatics 25, 1754-60 (2009).
40. Li, H. et al. The Sequence Alignment/Map format and SAMtools. Bioinformatics 25, 2078-9 (2009).
41. Kong, A. et al. Fine-scale recombination rate differences between sexes, populations and individuals. Nature 467, 1099-103.

TABLE 1
The Icelandic sick sinus syndrome study population

| | SSS | | Controls | |
|---|---|---|---|---|
| | Men | Women | Men | Women |
| N | 408 | 384 | 16,162 | 21,423 |
| Year | 1926.3 (10.8) | 1926.3 (13.1) | 1947.1 (22.1) | 1947.7 (21.0) |
| of | | | | |
| birth | | | | |
| (SD) | | | | |

SSS = sick sinus syndrome,
N = number,
SD = standard deviation.

TABLE 2
Association of R721W and rs365990[G] with ECG measures

| | | R721W | | | rs365990[G] | | |
|---|---|---|---|---|---|---|---|
| Phenotype | N | Effect (95% CI) | | P | Effect (95% CI) | | P |
| Heart rate | 12,135 | −5.05 (−8.11, −1.99) | bpm | 0.0030 | −0.91 (−1.30, −0.52) | bpm | 2.8e−05 |
| PR interval | 11,759 | 7.60 (2.90, 12.31) | ms | 0.0074 | 1.08 (0.47, 1.68) | ms | 0.0031 |
| QRS duration | 12,135 | 0.54 (−2.49, 3.57) | ms | 0.76 | −0.24 (−0.63, 0.15) | ms | 0.29 |
| QT interval | 12,135 | −0.28 (−5.41, 4.85) | ms | 0.92 | −0.61 (−1.26, 0.04) | ms | 0.093 |

Joint estimates of the association of the previously published rs365990[G] and R721W with EKG measures excluding known sick sinus syndrome and pacemaker cases. R721W occurs on the background of the G allele of rs365990.
N = number,
bpm = beats per minute,
ms = milliseconds.

TABLE 3
Refinement of the sick sinus syndrome association with R721W

| | R721W | |
|---|---|---|
| Phenotype status | carrier | Not R721W carrier |
| Sick sinus syndrome | 40 | 4 |
| Controls | 33 | 53 |

Among carriers of rs28730774[T]: 91% of sick sinus syndrome carriers also carry R721W while only 38% of controls also carry R721W (P = 2.9 × 10⁻⁹).

TABLE 4
Association of R721W with several cardiovascular diseases

| | Including known SSS cases | | | | Excluding known SSS cases | | | |
|---|---|---|---|---|---|---|---|---|
| Phenotype | N cases | N ctrls | OR (95% CI) | P | N cases | N ctrls | OR (95% CI) | P |
| Sick sinus syndrome | 776 | 36,955 | 12.53 (8.08, 19.44) | 1.5e−29 | — | — | — | — |
| Pacemaker implantation | 906 | 36,825 | 10.17 (6.56, 15.77) | 3.6e−25 | 295 | 36,660 | 1.35 (0.30, 6.16) | 0.7 |
| Atrial fibrillation | 2,318 | 35,413 | 2.39 (1.54, 3.71) | 0.0001 | 2,020 | 34,935 | 2.03 (1.15, 3.58) | 0.015 |
| Atrioventricular block | 254 | 37,477 | 2.80 (0.97, 8.10) | 0.057 | 177 | 36,778 | 1.17 (0.15, 9.31) | 0.88 |
| Sudden cardiac death | 327 | 37,404 | 1.63 (0.55, 4.80) | 0.38 | 293 | 36,662 | 2.50 (0.79, 7.90) | 0.12 |
| Aortic stenosis | 565 | 37,166 | 3.11 (1.51, 6.38) | 0.002 | 497 | 36,458 | 2.48 (0.93, 6.60) | 0.069 |
| Thoracic aortic aneurysm | 55 | 37,676 | 7.85 (1.68, 36.66) | 0.0088 | 53 | 36,902 | 11.68 (2.33, 58.57) | 0.0028 |
| Abdominal aortic aneurysm | 438 | 37,293 | 0.90 (2.90, 0.28) | 0.86 | 414 | 36,541 | 1.38 (0.39, 4.80) | 0.62 |
| Heart failure | 654 | 37,077 | 3.01 (1.52, 5.95) | 0.0016 | 563 | 36,392 | 1.51 (0.50, 4.52) | 0.46 |
| Coronary artery disease | 6,683 | 31,048 | 1.11 (0.78, 1.58) | 0.57 | 6,274 | 30,681 | 1.07 (0.71, 1.62) | 0.75 |

This table shows association between R721W and several cardiovascular diseases in Icelandic case-control sample sets, both including and excluding known cases of sick sinus syndrome (SSS).
N = number,
ctrls = controls.

1. A method of determining a susceptibility to a condition selected from Sick Sinus Syndrome, Atrial Fibrillation, Pacemaker implantation and Thoracic aortic aneurysm, the method comprising:
analyzing a nucleic acid sample obtained from a human individual with respect to at least one allele of the human MYH6 gene in the human individual;
and
determining an increased susceptibility to the condition for the human individual from the presence of the at least one MYH6 allele in the sample, or determining a decreased susceptibility to the condition in the individual from the absence of the at least one MYH6 allele in the sample.

2. The method of claim 1, wherein the condition is Sick Sinus Syndrome.

3-4. (canceled)

5. The method of claim 1, wherein the analysis of the nucleic acid comprises at least one procedure selected from:
(i) amplification of nucleic acid from the nucleic acid sample;
(ii) hybridization assay using a nucleic acid probe and nucleic acid from the nucleic acid sample;
(iii) hybridization assay using a nucleic acid probe and nucleic acid obtained by amplification of the nucleic acid sample, and
(iv) nucleic acid sequencing.

6-7. (canceled)

8. The method of claim 1, wherein the at least one allele encodes a defective MYH6 protein.

9. The method of claim 8, wherein the at least one allele encodes a missense substitution, a nonsense substitution or a truncation in a MYH6 protein with sequence as set forth in SEQ ID NO:3.

10. The method of claim 8, wherein the defective MYH6 protein has an impaired function selected from the group consisting of: an impaired converter domain, an impaired motor domain, an impaired lever arm domain, an impaired dimerization domain, an impaired alpha helix domain and an impaired myosin_N domain.

11. The method of claim 10, wherein the impaired function results in an impaired activity of the domain.

12-13. (canceled)

14. The method of claim 1, wherein the at least one allele of the MYH6 gene comprises the A allele of marker SG14S1131.

15. A method of determining whether an individual is at increased risk of developing Sick Sinus Syndrome, the method comprising steps of
obtaining a biological sample containing nucleic acid from the individual;
determining, in the biological sample, nucleic acid sequence about the MYH6 gene; and
comparing the sequence information to the wild-type sequence of MYH6 (SEQ ID NO:2);
wherein an identification of a mutation in MYH6 in the individual is indicative that the individual is at increased risk of developing Sick Sinus Syndrome.

16. The method of claim 15, wherein the mutation is a missense mutation, a nonsense mutation or a frameshift mutation in MYH6.

17. The method of claim 15, wherein the mutation results in a MYH6 defect selected from the group consisting of:
(a) premature truncation or frameshift of an encoded MYH6 protein, relative to the MYH6 amino acid sequence set forth in SEQ ID NO:3;
(b) expression of a MYH6 protein with reduced activity compared to a wild-type MYH6 protein (SEQ ID NO:3), wherein the activity is at least one MYH6 activity selected from:
(i) MYH6 dimerization; and
(ii) MYH6 motor activity;
(c) reduced expression of MYH6 protein, compared to wild-type MYH6,
wherein mutant alleles indicative of the defect are associated with increased susceptibility to Sick Sinus Syndrome.

18. A method of determining a susceptibility to Sick Sinus Syndrome, the method comprising:
analyzing a biological sample obtained from a human individual to obtain amino acid sequence data about at least one encoded MYH6 protein in a human individual;
analyzing the amino acid sequence data to determine whether at least one amino acid substitution predictive of increased susceptibility of Sick Sinus Syndrome is present; and
determining an increased susceptibility to Sick Sinus Syndrome for the individual from the presence of the at least one amino acid substitution in the at least one MYH6 protein, or determining a decreased susceptibility to Sick Sinus Syndrome for the individual from the absence of the at least one amino acid substitution.

19-20. (canceled)

21. The method of claim 18, wherein the amino acid sequence data is obtained using a method that comprises at least one procedure selected from:
(i) an antibody assay; and
(iii) protein sequencing.

22. (canceled)

23. The method of claim 18, wherein the amino acid substitution is an arginine to tryptophan substitution at position 721 in an MYH6 protein with sequence as set forth in SEQ ID NO:3.

24. The method of claim 18, further comprising the step of preparing a report containing results from the determination, wherein said report is written in a computer readable medium, printed on paper, or displayed on a visual display.

25. The method of claim 1, further comprising reporting the susceptibility to at least one entity selected from the group consisting of the individual, a guardian of the individual, a genetic service provider, a physician, a medical organization, and a medical insurer.

26-28. (canceled)

29. A method of selecting a human individual for treatment for Sick Sinus Syndrome, the method comprising:
(i) determining whether the individual has at least one symptom selected from the group consisting of
(a) sinus bradycardia;
(b) sinus arrest;
(c) chronotrophic incompetence;
(d) tachycardia; and
(e) atrial fibrillation;
(ii) analyzing sequence information about the individual for at least one allele of the human MYH6 gene, wherein at least one allele is predictive of increased susceptibility of Sick Sinus Syndrome in humans;
wherein the individual is selected for treatment for Sick Sinus Syndrome based on the presence of at least one symptom as listed in (i) and a determination of the presence of the at least one allele.

30. The method of claim 29, wherein the at least one allele comprises the A allele of marker SG14S1131.

31-40. (canceled)

41. A computer-readable medium having computer executable instructions for determining susceptibility to Sick Sinus Syndrome in a human individual, the computer readable medium comprising:
sequence data identifying at least one allele of at least one polymorphic marker in the individual;
a routine stored on the computer readable medium and adapted to be executed by a processor to determine risk of developing Sick Sinus Syndrome for the at least one polymorphic marker;
wherein the at least one polymorphic marker is a marker in the human MYH6 gene, or an amino acid substitution in an encoded MYH6 protein, that is predictive of susceptibility of Sick Sinus Syndrome in humans.

42. The computer-readable medium of claim 41, wherein the marker in the human MYH6 gene is SG14S1131.

43. The computer-readable medium of claim 41, wherein the amino acid substitution is an arginine to tryptophan substitution at position 721 in an encoded MYH6 protein with sequence as set forth in SEQ ID NO:1.

44. An apparatus for determining a susceptibility to Sick Sinus Syndrome in a human individual, comprising:
a processor;
a computer readable memory having computer executable instructions adapted to be executed on the processor to analyze information for at least one human individual with respect to at least one marker in the human MYH6 gene that is predictive of susceptibility to Sick Sinus Syndrome in humans, or at least one amino acid substitution in an encoded MYH6 protein, and generate an output based on the marker or amino acid information, wherein the output comprises at least one measure of susceptibility to Sick Sinus Syndrome for the human individual.

45. The apparatus of claim 44, wherein the marker information comprises nucleic acid sequence data identifying at least one allele of the at least one marker in the genome of the individual.

46. The apparatus according to claim 44, wherein the at least one marker is SG14S1131.

47. The apparatus of claim 44, wherein the amino acid substitution is an arginine to tryptophan substitution at position 721 in an MYH6 protein with sequence as set forth in SEQ ID NO: 3.

48. A system for identifying susceptibility to a condition selected from Sick Sinus Syndrome, Atrial Fibrillation, Pacemaker implantation and Thoracic aortic aneurysm in a human subject, the system comprising:
at least one processor;
at least one computer-readable medium;
a susceptibility database operatively coupled to a computer-readable medium of the system and containing population information correlating the presence or absence of one or more alleles of the human MYH6 gene and susceptibility to the condition in a population of humans;
a measurement tool that receives an input about the human subject and generates information from the input about the presence or absence of at least one mutant allele in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to the condition for the human subject.

49. The system according to claims 48, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to the condition for the subject.

50. The system according to claim 48, wherein the condition is Sick Sinus Syndrome.

51. The system according to claim 48, wherein the at least one mutant MYH6 allele is indicative of a MYH6 defect selected from the group consisting of:
(a) a missense substitution, a nonsense substitution or a truncation in a MYH6 protein with sequence as set forth in SEQ ID NO:3;
(b) a MYH6 defect resulting in an expressed MYH6 protein with an impaired function selected from the group consisting of: an impaired converter domain, an impaired motor domain, an impaired lever arm domain, an impaired dimerization domain, an impaired alpha helix domain or an impaired myosin_N domain,
wherein mutant alleles indicative of the defect are associated with increased susceptibility to the condition.

52. The system according to claim 48, wherein the measurement tool comprises a tool stored on a computer-readable medium of the system and adapted to be executed by a processor of the system to receive a data input about a subject and determine information about the presence or absence of the at least one mutant allele in a human subject from the data.

53. The system according to claim 52, wherein the data is genomic sequence information, and the measurement tool comprises a sequence analysis tool stored on a computer readable medium of the system and adapted to be executed by a processor of the system to determine the presence or absence of the at least one mutant allele from the genomic sequence information.

54. The system according to claim 48, wherein the input about the human subject is a biological sample from the human subject, and wherein the measurement tool comprises a tool to identify the presence or absence of the at least one mutant allele in the biological sample, thereby generating information about the presence or absence of the at least one mutant allele in a human subject.

55. The system according to claim 54, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one mutant allele based on the detection data.

56. The system according to claim 55, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one mutant allele based on the nucleotide sequence information.

57. The system according to claim 48, further comprising:
a medical protocol database operatively connected to a computer-readable medium of the system and containing information correlating the presence or absence of the at least one mutant allele and medical protocols for human subjects at risk for the condition; and
a medical protocol routine, operatively connected to the medical protocol database and the analysis routine, stored on a computer-readable medium of the system, and adapted to be executed on a processor of the system, to compare the conclusion from the analysis routine with respect to susceptibility to the condition for the subject and the medical protocol database, and generate a protocol report with respect to the probability that one or more medical protocols in the database will:
reduce susceptibility to the condition; or
delay onset of the condition; or
increase the likelihood of detecting the condition at an early stage to facilitate early treatment.

58. The system according to claim 49, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

59. The system according to claim 58, wherein the communication expresses the susceptibility to the condition in terms of odds ratio or relative risk or lifetime risk.

60. The system according to claim 58, wherein the communication further includes a protocol report.

61. The system according to claim 48, wherein the susceptibility database further includes information about at least one parameter selected from the group consisting of age, sex, ethnicity, race, medical history, weight, diabetes status, blood pressure, family history of the condition, and smoking history in humans and impact of the at least one parameter on susceptibility to the condition.

62. A system for assessing or selecting a treatment protocol for a subject diagnosed with a condition selected from Sick Sinus Syndrome, Atrial Fibrillation, Pacemaker implantation and Thoracic aortic aneurysm, comprising:
at least one processor;
at least one computer-readable medium;
a medical treatment database operatively connected to a computer-readable medium of the system and containing information correlating the presence or absence of at least one mutant MYH6 allele and efficacy of treatment regimens for the condition;
a measurement tool to receive an input about the human subject and generate information from the input about the presence or absence of the at least one mutant MYH6 allele in a human subject diagnosed with the condition; and
a medical protocol tool operatively coupled to the medical treatment database and the measurement tool, stored on a computer-readable medium of the system, and adapted to be executed on a processor of the system, to compare the information with respect to presence or absence of the at least one mutant MYH6 allele for the subject and the medical treatment database, and generate a conclusion with respect to at least one of:
the probability that one or more medical treatments will be efficacious for treatment of the condition for the patient; and
which of one or more medical treatments for the condition will be more efficacious for the patient.

63. The system according to claim 62, wherein the measurement tool comprises a tool stored on a computer-readable medium of the system and adapted to be executed by a processor of the system to receive a data input about a subject and determine information about the presence or absence of the at least one mutant MYH6 allele in a human subject from the data.

64. The system according to claim 63, wherein the data is genomic sequence information, and the measurement tool comprises a sequence analysis tool stored on a computer readable medium of the system and adapted to be executed by a processor of the system to determine the presence or absence of the at least one mutant MYH6 allele from the genomic sequence information.

65. The system according to claim 62, wherein the input about the human subject is a biological sample from the human subject, and wherein the measurement tool comprises a tool to identify the presence or absence of the at least one mutant MYH6 allele in the biological sample, thereby generating information about the presence or absence of the at least one mutant MYH6 allele in a human subject.

66. The system according to claim 62, further comprising a communication tool operatively connected to the medical protocol routine for communicating the conclusion to the subject, or to a medical practitioner for the subject.

67. The system according to claim 66, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

68. A method of using a nucleic acid sample isolated from a human individual to calculate a risk for Sick Sinus Syndrome, atrial fibrillation, pacemaker implantation or thoracic aortic aneurysm, the method comprising:
analyzing polymorphic marker SG14S1131 in the nucleic acid sample and determining that an A allele of polymorphic marker SG14S1131 is present in the nucleic acid sample, and
calculating a risk score for Sick Sinus Syndrome, atrial fibrillation, pacemaker implantation or thoracic aortic aneurysm in the individual that includes a relative risk (RR) or an odds ratio (OR) of at least 2.0 attributed to allele A of the marker(s) being present in the nucleic acid sample from the individual, using an apparatus comprising:
a computer readable memory;
a processor; and
a routine stored on the computer readable memory;
wherein the routine is adapted to be executed on the processor to analyze genotype data with respect to the at least one polymorphic marker and generate an output based on the genotype data, wherein the output comprises a risk score for the human individual with respect to susceptibility to Sick Sinus Syndrome, atrial fibrillation, pacemaker implantation or thoracic aortic aneurysm.

69. The method of claim 68, wherein the analyzing of the polymorphic marker in the nucleic acid sample is performed using a process selected from whole genome sequencing, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE), denaturing gradient gel electrophoresis (DGGE), mobility shift analysis, restriction enzyme analysis, heteroduplex analysis, chemical mismatch cleavage (CMC), RNase protection assays, allele-specific PCR, and direct manual or automated sequencing.

70. The method of claim 68, further comprising reporting the risk score to at least one entity selected from the group consisting of the individual, a guardian of the individual, a genetic service provider, a physician, a medical organization, and a medical insurer.

71. The method of claim 68, further comprising performing a long term cardiac rhythm monitoring test on the individual determined to have the increased susceptibility to Sick Sinus Syndrome.
